(19) United States
(12) Patent Application Publication
(10) Pub. No.: US 2004/0063681 A1
(43) Pub. Date: Apr. 1, 2004

(54) Methods for using gold (III) complexes as anti-tumor and anti-HIV agents

(76) Inventor: Chi-Ming Che, Hong Kong (CN)

Correspondence Address: JONES DAY, 222 EAST 41ST STREET, NEW YORK, NY, 10017, US

(21) Appl. No.: 10/663,894

(22) Filed: Sep. 16, 2003

Related U.S. Application Data
(60) Provisional application No. 60/411,423, filed on Sep. 16, 2002.

(51) Int. Cl. A61K 31/555

(52) U.S. Cl. 514/185; 514/410

(57) ABSTRACT

Disclosed are pharmaceutical compositions comprising gold(III) complexes of porphyrins, Schiff-bases, bis(pyridyl)carboxamides and bis(pyridyl)sulfonamides. Also disclosed are methods for using the pharmaceutical compositions as anti-tumor and anti-HIV agents.

This application claims the benefit of U.S. Provisional Application No. 60/411,423, filed Sep. 16, 2002, the entire disclosure being incorporated herein by reference.

### FIELD OF THE INVENTION

The invention is directed to the use of gold(III) complexes as anti-tumor and anti-HIV agents.

### BACKGROUND OF THE INVENTION

The success of cisplatin as an anti-tumor drug has stimulated considerable interest in using metal complexes as modem therapeutic, diagnostic and radiopharmaceutical agents (see Chem. Rev. 99: whole issue (1999)). In view of the emergence of cisplatin-resistant cancer strains and severe toxicity of cisplatin, development of new metal-based drugs with less toxic side effects and ability to overcome the drug resistance are being actively pursued by various research groups worldwide (see Clarke et al., Topiccs in Biological Inorganic Chemistry: Metallopharmaceuticals I, DNA Interactions, Springer, Berlin 1999).

Gold compounds have long been used as medicinal agents for rheumatoid arthritis; notable examples include auranofin and myocrysin (see Shaw, III, in Gold-Progress in Chemistry, Biochemistry and Technology; Schmidbaur, H., Ed.; Wiley: N.Y., 1999, 259). The discovery by Lorber and co-workers in 1979 that auranofin could inhibit proliferation of HeLa cells in culture had stimulated an extensive interest in the pharmacological potential of gold compounds (see Simon et al., Cancer 44:1965 (1979)), and a series of auranofin derivatives and diphenylphosphinogold(I) complexes was evaluated intensively for in vitro and in vivo anti-tumor activities during the 1980s (see Shaw, III, in Uses of Inorganic Chemistry in Medicine, Farrell, N. P. Ed., 1999, Royal Society of Chemistry: Cambridge, Chapter 3, page 27). Unfortunately, these gold(I) complexes exhibit severe cardiotoxicity in animal studies that rendered them unfavorable for clinical applications. (See, e.g., Hoke et al., Toxicol. Appl. Pharmacol. 100:293 (1989); and Tiekink, Critical Rev. Oncology/Hematology, 42:225-248 (2002)).

Being isostructural and isoelectronic to platinum(II), the Au(III) ion forms square-planar complexes and, therefore, has long been anticipated to be a promising anti-tumor agent. See Pieper et al., in Topics in Biological Inorganic Chemistry: Metallopharmaceuticals I, DNA Interactions, Clarke, M. J.; Sadler, P. J. Eds.; Springer: Berlin, 1999; pages 171-199. In contrast to the extensive reports on the biological properties of gold(I) complexes, studies on the anti-tumor and cytotoxic properties of gold(III) complexes are sparse in the literature. The major problem for development of anti-tumor gold(III) compounds is that gold(III) complexes have high redox potential and readily undergo reduction to gold(I) and colloidal gold in physiological buffer solutions.

To date, two major classes of anti-tumor gold(III) complexes (depicted in Scheme 1) have been extensively studied. The class I compounds encompass those gold(III) complexes containing one, two or three labile ligand(s) (e.g., -halide, -SCN) and a mono-/bi-/tridentate auxiliary ligand. For the class II compounds, the four-coordinated gold(III) atom without labile ligand(s) is coordinated to some chelating polyamine ligands. According to the literature, all these gold(III) compounds have limited stability in physiological buffer solutions; especially they are readily reduced by mild reductants such as sodium thiosulfate and glutathione to give colloidal gold (see Messori et al., J. Med. Chem. 43:3541 (2000)). To our knowledge, there has been limited success in preparing potent anti-tumor gold(III) compounds that are stable in physiologically relevant conditions. Scheme 1 shows selected literature examples of anti-tumor gold(III) complexes, where X=I⁻, Br⁻, Cl⁻, SCN⁻ or CH₃CO₂⁻.

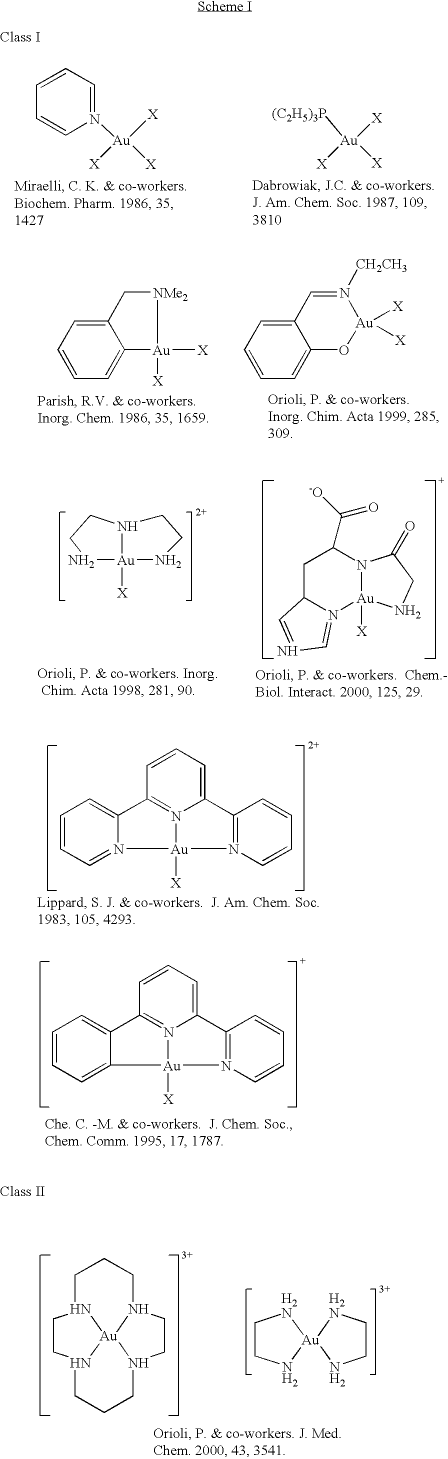

Fleischer et al., Inorg. Nucl. Chem. Lett. 5:373 (1969) first reported the synthesis of [Au^{III}(TPP)]AuCl₄. See also Falk, J. E. Porphyrins and Metalloporphyrins; Elsevier: Amsterdam, 1964. Jamin et al., Inorganica Chimica Acta 27:135 (1978) discloses other gold(III) porphyrin derivatives including [Au^{III}(mesoporphyrin IX)]AuCl₄; and Abou-Gamra et al., J. Chem. Soc., Faraday Trans. 2, 82:2337 (1986) discloses [Au^{III}(meso-tetrakis(N-methyl-4-pyridyl)porphyrin)]Cl₅. These procedures were employed for the preparation of the gold(III) porphyrin complexes reported in this work. Abou-Gamra et al., J. Chem. Soc., Faraday Trans. 2, 82:2337 (1986) discloses the electrochemistry of gold(III) porphyrins in aqueous media, while Jamin et al., Inorganica Chimica Acta 27:135 (1978) discloses the electrochemistry of gold(III) porphyrins in non-aqueous media. The gold(III) porphyrins were reported to undergo porphyrin-centered redox reactions without reduction of the gold(III) center. However, the biological properties of the gold(III) porphyrin complexes, especially their anti-tumor and cytotoxic properties, are completely unknown in the literature.

Metalloporphyrins are an important class of molecules that form the active sites of numerous proteins for a variety of biological functions including dioxygen transport and storage (hemoglobin, myoglobin), dioxygen activation (cytochrome P-450), electron transport (cytochrome c, cytochrome oxidase) and energy conversion (chlorophyll) (see Milgrom, L. R. The Colours of Life; Oxford University Press Inc.: New York, 1997; and Kadish, K. M. et al.; The Porphyrin Handbook; Eds.; Academic Press: San Diego, 2000). Pioneered by Fiel and Pasterneck, interaction of metalloporphyrins with DNA has been a subject of intense investigation. Metalloporphyrins have been used as probes for nucleic acid structures and dynamics (see Bennett et al., Proc. Natl. Acad. Sci. USA 97:9476 (2000); Guliaev et al., Biochemistry 38:15425 (1999); Lipscomb et al., Biochemistry 35:2818 (1996); and Marzilli et al., J. Am. Chem. Soc. 114:7575 (1992)) and as reagents for DNA footprinting analysis (see Mestre et al., Biochemistry 35:9140 (1996)). Porphyrins and their derivatives have been used in photodynamic cancer therapy; however, few porphyrins and their metal complexes are known to exert significant cytotoxic effects to human cells/tissues without photoactivation. See Hill et al., Proc. Natl. Acad. Sci. USA 92:12126 (1995) and international publication No. WO 00/12512 A1. Recently, Hurley and co-workers found that some cationic meso-tetrakis(N-methylpyridyl)porphyrins could stabilize G-quadruplex DNA and act as a potential inhibitor of human telomerase for anti-cancer treatment (see Han et al., J. Am. Chem. Soc. 121:3561-3570 (1999)). However, there is no evidence that these porphyrins would induce cancer cell death according to Hurley's report.

Human Immunodeficiency Virus type 1 (HIV-1) Reverse Transcriptase (RT) is an important drug target for clinical treatment of Acquired Immunodeficiency Syndrome (AIDS) (see Mitsuya et al., Science 249:1533 (1990)). There are two major classes of clinically used HIV-I reverse transcriptase inhibitors: (1) nucleoside RT inhibitors [NRTIs such as 3′-azido-2′,3′-dideoxythymidine (AZT), 2′,3′-dideoxyinosine (ddI), and 2′-deoxy-3′-thiacytidine (3TC)], and (2) non-nucleoside RT inhibitors (NNRTIs such as nevirapine, delavirdine and efavirenz) (see Anti-AIDS Drug Development: Challenges, Strategies and Prospects (P. Mohan and M. Baba eds. 1995, chapter 11, page 239). NRTIs bind to the normal deoxynucleoside triphosphate (dNTP) substrate-binding site and inhibit HIV replication by terminating DNA chain elongation (see Jacobo-Molina et al., Proc. Natl. Acad. Sci. USA 90:6320 (1991); and Huang et al., Science 282:1669 (1998)). NNRTIs are believed to inhibit the chemical step of polymerization by binding to a distinct site nearby the polymerase active site. Although the current chemotherapeutic therapy generally involves combinations of both NRTIs and NNRTIs in order to delay emergence of resistance, negative impacts of the treatment including drug toxicity, generation of multidrug-resistant phenotypes and presence of latent virus reservoirs have been reported (see Cohen, Science 277:32 (1997)).

Some synthetic metalloporphyrins are reported to inhibit HIV-I reverse transcriptase activity in vitro (see Paterson et al, J. Biol. Chem. 274:1549 (1999); and Staudinger et al., Proc. Assoc. Am. Phys. 108:47 (1996)).

Tetradentate Schiff-base and bis(pyridyl)carboxamide ligands reportedly form complexes with gold(III) ions. See Barnholtz et al., Inorg. Chem. 40:972 (2001); Dar et al., J. Chem. Soc., Dalton Trans. 1907 (1992); Banerjee et al., Ind. J. Chem. 23A:555 (1984); Murray et al., J. Organomet. Chem. 61:451 (1973); and Inazu, Bull. Chem. Soc. Jpn. 39:1065 (1966). Yet, the anti-tumor and cytotoxicities of these complexes remain largely unexplored.

There remains a need, however, for effective anti-cancer agents and anti-HIV agents.

Citation of any reference in this Section of the application is not an admission that the reference is prior art to the application.

### SUMMARY OF THE INVENTION

The invention relates to methods for using gold(III)porphyrin complexes, gold(III)Schiff-base complexes and gold(III)carboxamide complexes (“Gold(III) Complexes”) as anti-tumor and anti-HIV agents.

In one embodiment, the invention relates to a method for induction of apoptosis of cancer cells comprising administering to a patient in need thereof a composition comprising an effective amount of a gold(III) complex of formula (I):

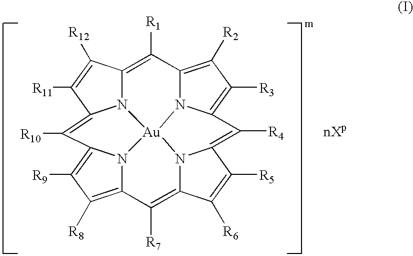

or a pharmaceutically acceptable salt thereof, wherein:

R₁, R₄, R₇ and R₁₀ are each independently —H, -halo, —(C₁-C₆)alkyl or —O(C₁-C₆)alkyl, -(6-membered)aryl or -(5 to 10-membered)heteroaryl, each of which may be substituted with one or more -halo, —(C₁-C₆)alkyl, —O(C₁-C₆)alkyl, —OSO₂ or —NO₂;

R₂, R₃, R₅, R₆, R₈, R₉, R₁₁ and R₁₂ are each independently —H, —(C₁-C₆)alkyl which may be substituted with one or more —C(O)OR₁₃, -halo or ═O groups; R₁₃ is —(C₁-C₆)alkyl;

each X^{p} is independently a pharmaceutically acceptable counter-ion;

m is an integer ranging from −3 to 5;

p is an integer ranging from −3 to 3;

n is equal to the absolute value of m/p; and

a pharmaceutically acceptable carrier.

In another embodiment, the invention relates to a method for induction of apoptosis of cancer cells comprising administering to a patient in need thereof a composition comprising an effective amount of a gold(III) complex of formula (II):

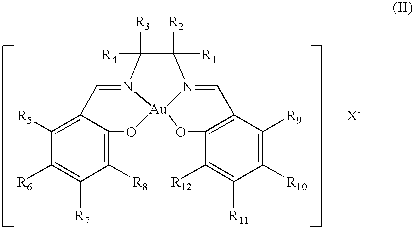

or a pharmaceutically acceptable salt thereof, wherein:

R₁-R₁₂ are each independently —H, -halo, —(C₁-C₆)alkyl or —O(C₁-C₆)alkyl which may be substituted with one or more —O(C₁-C₆)alkyl or -halo;

X is a counter-anion; and

a pharmaceutically acceptable carrier.

In another embodiment, the invention relates to a method for induction of apoptosis of cancer cells comprising administering to a patient in need thereof a composition comprising an effective amount of a gold(III) complex of formula (III):

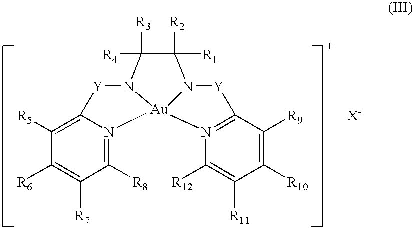

or a pharmaceutically acceptable salt thereof, wherein:

(a) R₁-R₁₂ are each independently —H, -halo, —(C₁-C₆)alkyl —O(C₆)alkyl which may be substituted with one or more —O(C₁-C₆)alkyl or -halo; or

(b) R₁ and R₄ are absent; and R₂ and R₃ together form a 6-membered aryl ring of formula

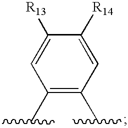

Y is

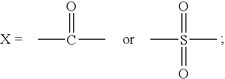

R₁₃ and R₁₄ are each —H or -halo;

X is a counter-anion; and

a pharmaceutically acceptable carrier.

The invention relates to methods for inhibition of reverse transcriptase of Human Immunodeficiency virus-1.

In one embodiment, the invention relates to a method for inhibition of reverse transcriptase of Human Immunodeficiency virus-1 comprising administering to a patient in need thereof an effective amount of a gold(III) complex of formula (1), or a pharmaceutically acceptable salt thereof, wherein, and a pharmaceutically acceptable carrier.

In another embodiment, the invention relates to a method for inhibition of reverse transcriptase of Human Immunodeficiency virus-1 comprising administering to a patient in need thereof an effective amount of a gold(III) complex of formula (II), or a pharmaceutically acceptable salt thereof, wherein, and a pharmaceutically acceptable carrier.

In another embodiment, the invention relates to a method for inhibition of reverse transcriptase of Human Immunodeficiency virus-1 comprising administering to a patient in need thereof an effective amount of a gold(III) complex of formula (III), or a pharmaceutically acceptable salt thereof, wherein, and a pharmaceutically acceptable carrier.

The invention also relates to pharmaceutical compositions comprising a gold(III)complex of the invention and a pharmaceutically acceptable carrier.

In one embodiment, the invention relates to a pharmaceutical composition comprising an effective amount of a complex of formula (I) and a pharmaceutically acceptable carrier.

In one embodiment, the invention relates to a pharmaceutical composition comprising an effective amount of a complex of formula (II) and a pharmaceutically acceptable carrier.

In one embodiment, the invention relates to a pharmaceutical composition comprising an effective amount of a complex of formula (III) and a pharmaceutically acceptable carrier.

### BRIEF DESCRIPTION OF THE DRAWINGS

FIG. 1 shows the UV-visible spectra in dichloromethane of (a) free base H₂TPP and (b) Complex 1a.

FIG. 2 shows the ORTEP drawing of [Au(TPP)]OCl₄, the perchlorate salt analog of Complex 1a, with atom-numbering scheme. Hydrogen atoms and the perchlorate ion are omitted for clarity. Thermal ellipsoids are drawn at the 30% probability level.

FIG. 3 shows the UV-visible spectrum of Complex 1a (2.5 μM) in Tris buffer/MeCN (19:1).

FIG. 4 shows the UV-visible spectra of Complex 1a (2.5 μM) in 2 mM GSH Tris buffer/MeCN (19:1) (a) time=0 h and (b) time=48 h.

FIG. 5 shows the cyclic voltammogram of Complex 1a recorded in acetonitrile (0.1 M Bu₄NPF₆). Working electrode=glassy carbon, counter-electrode=platinum wire, reference electrode=Ag/AgNO₃ (0.1 M in MeCN), scan rate=200 mVs⁻¹

FIG. 6 shows the linear dual-parameter Hammett correlation (R=0.99, slope=1.00) for a series of para-substituted Complexes 1c, 1b, 1a, 1d and 1e.

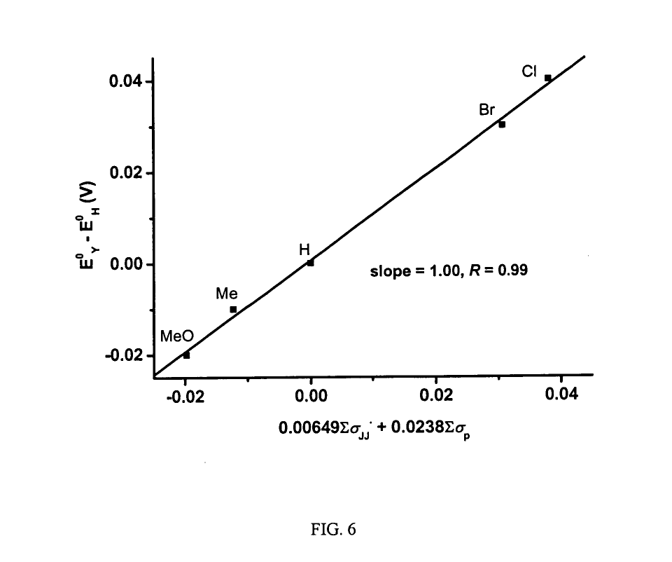

FIG. 7 shows the cytoxiticity profiles of (a) human nasopharyngeal carcinoma (SUNE1) and (b) its cisplatin-resistant variant (CNE1) toward Complexes 1a, 1f, and 1i. Graphs show the percentage of growth compared to control upon incubation of increasing amounts of the gold(III) complexes. For comparison, curves for KAu^{III}Cl₄ and Pt(NH₃)₂Cl₂ (cisplatin) are also shown.

FIG. 8 shows the electrophoresis of a 9.4 kbp plasmid (pDR2) in 1% (w/v) agarose gel after restriction enzyme (ApaI) digestion in the absence (lane A) and presence (lane C-H) of various compounds labeled with the [compound]/[bp]. Lane B is the undigested DNA.

FIG. 9 shows the UV-vis spectral changes of Complex 1a in Tris buffer with increasing concentration of ctDNA: (a) r=0, (b) r=0.21, (c) r=0.41, (d) r=0.62, (e) r=0.83, (f) r=1.03, (g) r=1.24, and (h) r=1.45 at 292.8 K. Inset: plots of Aₒ/A vs [DNA]/[Complex [1a]and [ctDNA]/Δεₐₚ vs [ctDNA]. Absorbance was monitored at 410 nm.

FIG. 10 shows the relative viscosity of calf thymus DNA in the presence of ethidium bromide (●), Hoechst 33342 (▴) or Complex 1a (□), shown as a function of the binding ratio (r).

FIG. 11 shows the gel electrophoresis of 100-bp DNA in 2% (w/v) agarose gel showing the mobilities of the DNA (15.2 □M bp-1) in the absence (lane A & F) and the presence of ethidium bromide (lane B), Hoechst 33342 (lane C), and Complex 1a (lane D & E, labeled with the [compound]/[bp]).

FIG. 12 shows laser confocal micrographs of the HeLa cells treated with Complex 1a (0.5 μM) at time interval of (a) 0 h and (b) 15 h. Apoptotic cells are marked by a circle

FIG. 13 shows the results of apoptotic studies of HeLa cells using flow cytometry: Annexin V/propidium iodide assay. Flow cytometric results of (a) DMSO, 1.5% (v/v), 15 h; (b) straurosporine, 15 h; (c) Complex 1a, 6 h and (d) Complex 1a, 15 h. The corresponding plots of cell counts vs. annexin-V emission are shown in 13(e)-(h).

FIG. 14 shows the results of an HIV-1 RT inhibition study, comparing the activity of Complexes 1j, 1k, 2a and 3c with their corresponding free ligands [H₂TMPyP]⁴⁺, [H₂TPPS]⁴⁻, H₂(Salen) and H₂(N₄). FIG. 14 also includes comparative HIV-1 RT inhibition data for Au^{III}Cl₄, [(Ph₃P)Au^{I}Cl], Zn^{II}(PP) and [Zn_{II}(TMPyP)]⁴⁺. The concentration of the metal complexes and free ligands was 6 μM.

FIG. 15 shows the cytotoxicity profiles of the Complexes 1a, 1j, 1k, 2a and 3c toward human lung fibroblast cell line (CCD-19Lu) as a plot of % cell survival vs log[concentration of gold(III) complex]

FIG. 16 shows the results of an HIV-1 RT inhibition study using Complex 1j alone and in combination with AZT.

### DETAILED DESCRIPTION OF THE INVENTION

The present invention is directed to the use of Gold(III) Complexes as anti-tumor and anti-HIV agents.

It will be understood by those skilled in the art that the ligand and the cationic metal center may not form a charge neutral complex. For example, the net positive charge of the cationic metal may be greater than the absolute net negative charge of the deprotonated macrocyle ligand; or the net positive charge of the cationic metal may be less than the absolute net negative charge of the deprotonated macrocyle ligand. In such case, one or more counter-ions will be present to maintain charge neutrality between the ligand and the metal center. Accordingly, the phrase “pharmaceutically acceptable salt,” as used herein also includes salts formed from charged metal complex and the counter-ion.

As used herein, the phrase “counter-anion” refers to an anion associated with a positively charged Gold(III) Complex. Non-limiting examples of counter-anions include fluoride, chloride, bromide, iodide, sulfates and phosphates.

As used herein, the phrase “counter-cation” refers to a cation associated with a negatively charged Gold(III) Complex. Non-limiting examples of counter-cations include Na⁺ and K⁺.

As used herein, the term “TPP” means the dianions of meso-tetrakis(tetraphenyl)porphyrin.

As used herein, the term “GSH” means glutathione.

“-(6 to 10-membered)aryl means an aromatic ring of 6 to 10 members, including both mono- and bicyclic ring systems. Representative “-(6 to 10-membered)aryls include -phenyl, -naphthyl, and indenyl, which may be substituted or unsubstituted.

“-(5- to 10-membered)heteroaryl” means an aromatic heterocycle ring of 5 to 10 members, including both mono- and bicyclic ring systems, where at least one carbon atom of one or both of the rings is replaced with a heteroatom independently selected from nitrogen, oxygen, and sulfur. One or both of the -(5- to 10-membered)heteroaryl's rings contain at least one carbon atom. Representative -(5- to 10-membered)heteroaryls include -pyridyl, -furyl, -benzofuranyl, -thiophenyl, -benzothiophenyl, -quinolinyl, -pyrrolyl, -indolyl, -oxazolyl, -benzoxazolyl, -imidazolyl, -benzimidazolyl, -thiazolyl, -benzothiazolyl, -isoxazolyl, -pyrazolyl, -isothiazolyl, -pyridazinyl, -pyrimidinyl, -pyrazinyl, -triazinyl, -cinnolinyl, -phthalazinyl, -quinazolinyl and the like. The -(5- to 10-membered)heteroaryls may be substutited or unsubstituted.

“-(C₁-C₆)alkyl” means a saturated straight chain or branched non-cyclic hydrocarbon having from 1 to 6 carbon atoms. Representative saturated straight chain —(C₁-C₆)alkyls include -methyl, -ethyl, -n-propyl, -n-butyl, -n-pentyl, and -n-hexyl. Representative saturated branched —(C₁-C₆)alkyls include -isopropyl, -sec-butyl, -isobutyl, -tert-butyl, -isopentyl, -2-methylbutyl, -3-methylbutyl, -2,2-dimethylbutyl, -2,3-dimethylbutyl, -2-methylpentyl, -3-methylpentyl, -4-methylpentyl and the like.

As used herein, the phrase pharmaceutically acceptable carrier” means approved by a regulatory agency of the Federal or a state government or listed in the U.S. Pharmacopeia or other generally recognized pharmacopeia for use in animals, mammals, and more particularly in humans. Non-limiting examples of pharmaceutically acceptable carriers include liquids, such as water and oils, including those of petroleum, animal, vegetable, or synthetic origin. Water is a preferred vehicle when the compound of the invention is administered intravenously. Saline solutions and aqueous dextrose and glycerol solutions can also be employed as liquid vehicles, particularly for injectable solutions.

As used herein, the phrase “patient” refers to an animal. Non-limiting examples of animals include a cow, monkey, horse, sheep, pig, chicken, turkey, quail, cat, dog, mouse, rat, rabbit, and guinea pig, and more preferably a mammal, and most, preferably a human.

As used herein, the term “ligand” refers to an ion or molecule that binds to the Gold(III) Complexes of the invention.

As noted above, the present invention relates to compositions useful for the induction of apoptosis of cancer cells.

In one embodiment, the invention relates to a method for induction of apoptosis of cancer cells comprising administering to a patient in need thereof a composition comprising an effective amount of a gold(III) complex of formula (I) or a pharmaceutically acceptable salt thereof, wherein:

R₁, R₄, R₇ and R₁₀ are each independently —H, -halo, —(C₁-C₆)alkyl or —O(C₁-C₆)alkyl, -(6-membered)aryl or -(5 to 10-membered)heteroaryl, each of which may be substituted with one or more -halo, —(C₁-C₆)alkyl, —O(C₁-C₆)alkyl, —OSO₂ or —NO₂;

R₂, R₃, R₅, R₆, R₈, R₉, R₁₁ and R₁₂ are each independently —H, —(C₁-C₆)alkyl which may be substituted with one or more —C(O)OR₁₃, -halo or ═O groups;

R₁₃ is —(C₁-C₆)alkyl;

each X^{p} is independently a pharmaceutically acceptable counter-ion;

m is an integer ranging from −3 to 5;

p is an integer ranging from −3 to 3;

n is equal to the absolute value of m/p; and

a pharmaceutically acceptable carrier.

In another embodiment, the invention relates to a method for induction of apoptosis of cancer cells comprising administering to a patient in need thereof a composition comprising an effective amount of a gold(III) complex of formula (I) or a pharmaceutically acceptable salt thereof, wherein R₂, R₃, R₅, R₆, R₈, R₉, R₁₁, and R₁₂ are each —H.; X^{p} is Cl⁻; m is 1; n is 1; and a pharmaceutically acceptable carrier.

In another embodiment, the invention relates to a method for induction of apoptosis of cancer cells comprising administering to a patient in need thereof a composition comprising an effective amount of a gold(III) complex of formula (I) or a pharmaceutically acceptable salt thereof, wherein R₁, R₄, R₇ and R₁₀ are each -phenyl; R₂, R₃, R₅, R₆, R₈, R₉, R₁₁ and R₁₂ are each —H.; X^{p} is Cl⁻; m is 1; n is 1; and a pharmaceutically acceptable carrier.

In another embodiment, the invention relates to a method for induction of apoptosis of cancer cells comprising administering to a patient in need thereof a composition comprising an effective amount of a gold(III) complex of formula (I) or a pharmaceutically acceptable salt thereof, wherein R₁, R₄, R₇ and R₁₀ are each -4-methylphenyl; R₂, R₃, R₅, R₆, R₈, R₉, R₁₁ and R₁₂ are each —H.; X^{p} is Cl⁻; m is 1; an is 1; and a pharmaceutically acceptable carrier.

In another embodiment, the invention relates to a method for induction of apoptosis of cancer cells comprising administering to a patient in need thereof a composition comprising an effective amount of a gold(III) complex of formula (I) or a pharmaceutically acceptable salt thereof, wherein R₁, R₄, R₇ and R₁₀ are each -4-methoxyphenyl; R₂, R₃, R₅, R₆, R₈, R₉, R₁₁ and R₁₂ are each —H.; X^{p} is Cl⁻; m is 1; n is 1; and a pharmaceutically acceptable carrier.

In another embodiment, the invention relates to a method for induction of apoptosis of cancer cells comprising administering to a patient in need thereof a composition comprising an effective amount of a gold(III) complex of formula (I) or a pharmaceutically acceptable salt thereof, wherein R₁, R₄, R₇ and R₁₀ are each -4-bromophenyl; R₂, R₃, R₅, R₆, R₈, R₉, R₁₁ and R₁₂ are each —H.; X^{p} is Cl⁻; m is 1; n is 1; and a pharmaceutically acceptable carrier.

In another embodiment, the invention relates to a method for induction of apoptosis of cancer cells comprising administering to a patient in need thereof a composition comprising an effective amount of a gold(III) complex of formula (I) or a pharmaceutically acceptable salt thereof, wherein R₁, R₄, R₇ and R₁₀ are each -4-chlorophenyl; R₂, R₃, R₅, R₆, R₈, R₉, R₁₁ and R₁₂ are each —H.; X^{p} is Cl⁻; m is 1; n is 1; and a pharmaceutically acceptable carrier.

In another embodiment, the invention relates to a method for induction of apoptosis of cancer cells comprising administering to a patient in need thereof a composition comprising an effective amount of a gold(III) complex of formula (I) or a pharmaceutically acceptable salt thereof, wherein R₁, R₄, R₇ and R₁₀ are each -3,4,5-trimethoxyphenyl; R₂, R₃, R₅, R₅, R₈, R₉, R₁₁ and R₁₂ are each —H.; X^{p} is Cl⁻; m is 1;

n is 1; and a pharmaceutically acceptable carrier.

In another embodiment, the invention relates to a method for induction of apoptosis of cancer cells comprising administering to a patient in need thereof a composition comprising an effective amount of a gold(III) complex of formula (I) or a pharmaceutically acceptable salt thereof, wherein R₁, R₄, R₇ and R₁₀ are each -3,4,5-trifluorophenyl; R₂, R₃, R₅, R₆, R₈, R₉, R₁₁ and R₁₂ are each —H.; X^{p} is Cl⁻; m is 1; n is 1; and a pharmaceutically acceptable carrier.

In another embodiment, the invention relates to a method for induction of apoptosis of cancer cells comprising administering to a patient in need thereof a composition comprising an effective amount of a gold(III) complex of formula (I) or a pharmaceutically acceptable salt thereof, wherein R₁, R₄, R₇ and R₁₀ are each —H; R₂, R₃, R₅, R₆, R₈, R₉, R₁ and R₁₂ are each -ethyl; X^{p} is Cl⁻; m is 1; n is 1; and a pharmaceutically acceptable carrier.

In another embodiment, the invention relates to a method for induction of apoptosis of cancer cells comprising administering to a patient in need thereof a composition comprising an effective amount of a gold(III) complex of formula (I) or a pharmaceutically acceptable salt thereof, wherein R₁, R₄, R₇ and R₁₀ are each —H; and R₂ and R₁₁ are each -ethyl; R₃, R₅, R₉ and R₁₂ are each -methyl; R₆ and R₈ are each -methyl-3-propanoate; X^{p} is Cl⁻; m is 1; n is 1; and a pharmaceutically acceptable carrier.

In another embodiment, the invention relates to a method for induction of apoptosis of cancer cells comprising administering to a patient in need thereof a composition comprising an effective amount of a gold(III) complex of formula (I) or a pharmaceutically acceptable salt thereof, wherein R₁, R₄, R₇ and R₁₀ are each -4-(N-methyl)pyridinium; R₂, R₃, R₅, R₆, R₈, R₉, R₁₁ and R₁₂ are each —H; X^{p} is Cl⁻; m is 5; n is 5; and a pharmaceutically acceptable carrier.

In another embodiment, the invention relates to a method for induction of apoptosis of cancer cells comprising administering to a patient in need thereof a composition comprising an effective amount of a gold(III) complex of formula (I) or a pharmaceutically acceptable salt thereof, wherein R₁, R₄, R₇ and R₁₀ are each -4-sulfanatophenyl; R₂, R₃, R₅, R₆, R₈, R₉, R₁₁ and R₁₂ are each —H; X^{p} is Na⁺; m is +3; n is 3; and a pharmaceutically acceptable carrier.

In another embodiment, the invention relates to a method for induction of apoptosis of cancer cells comprising administering to a patient in need thereof a composition comprising an effective amount of a gold(III) complex of formula (II) or a pharmaceutically acceptable salt thereof, wherein:

R₁-R₁₂ are each independently —H, -halo, —(C₁-C₆)alkyl or —O(C₁-C₆)alkyl which may be substituted with one or more —O(C₁-C₆)alkyl or -halo;

X is a counter-anion; and

a pharmaceutically acceptable carrier.

In another embodiment, the invention relates to a method for induction of apoptosis of cancer cells comprising administering to a patient in need thereof a composition comprising an effective amount of a gold(III) complex of formula (II) or a pharmaceutically acceptable salt thereof, wherein R₁-R₄ are each —H; X is Cl⁻; and a pharmaceutically acceptable carrier.

In another embodiment, the invention relates to a method for induction of apoptosis of cancer cells comprising administering to a patient in need thereof a composition comprising an effective amount of a gold(III) complex of formula (II) or a pharmaceutically acceptable salt thereof, wherein R₁-R₁₂ are each —H; X is Cl; and a pharmaceutically acceptable carrier.

In another embodiment, the invention relates to a method for induction of apoptosis of cancer cells comprising administering to a patient in need thereof a composition comprising an effective amount of a gold(III) complex of formula (II) or a pharmaceutically acceptable salt thereof, wherein R₃, R₅—R₇ and R₉-R₁₀ are each —H; and R₄ and R₈ are each —Cl; X is Cl; and a pharmaceutically acceptable carrier.

In another embodiment, the invention relates to a method for induction of apoptosis of cancer cells comprising administering to a patient in need thereof a composition comprising an effective amount of a gold(III) complex of formula (II) or a pharmaceutically acceptable salt thereof, wherein R₁-R₅, R₇, R₉, R₁₁ are each —H; and R₆, R₈, R₁₀ and R₁₂ are each —Cl; X is Cl⁻; and a pharmaceutically acceptable carrier.

In another embodiment, the invention relates to a method for induction of apoptosis of cancer cells comprising administering to a patient in need thereof a composition comprising an effective amount of a gold(III) complex of formula (III) or a pharmaceutically acceptable salt thereof, wherein:

(a) R₁-R₁₂ are each independently —H, -halo, —(C₁-C₆)alkyl —O(C₆)alkyl which may be substituted with one or more —O(C₁-C₆)alkyl or -halo; or

(b) R₁ and R₄ are absent; and R₂ and R₃ together form a 6-membered aryl ring of formula

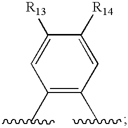

Y is

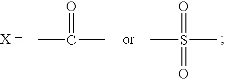

R₁₃ and R₁₄ are each —H or -halo;

X is a counter-anion; and

a pharmaceutically acceptable carrier.

In another embodiment, the invention relates to a method for induction of apoptosis of cancer cells comprising administering to a patient in need thereof a composition comprising an effective amount of a gold(III) complex of formula (III or a pharmaceutically acceptable salt thereof, wherein

Y is

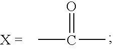

X is Cl⁻; and a pharmaceutically acceptable carrier.

In another embodiment, the invention relates to a method for induction of apoptosis of cancer cells comprising administering to a patient in need thereof a composition comprising an effective amount of a gold(III) complex of formula (III or a pharmaceutically acceptable salt thereof, wherein R₁-R₁₂ are each —H;

Y is

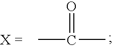

X is Cl⁻; and a pharmaceutically acceptable carrier.

In another embodiment, the invention relates to a method for induction of apoptosis of cancer cells comprising administering to a patient in need thereof a composition comprising an effective amount of a gold(III) complex of formula (III or a pharmaceutically acceptable salt thereof, wherein R₁-R₄ are each -methyl; R₅-R₁₂ are each —H;

Y is

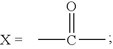

X is Cl⁻; and a pharmaceutically acceptable carrier.

In another embodiment, the invention relates to a method for induction of apoptosis of cancer cells comprising administering to a patient in need thereof a composition comprising an effective amount of a gold(III) complex of formula (III or a pharmaceutically acceptable salt thereof, wherein R₁ and R₄-R₁₂ are each —H; R₂ and R₃ are each -phenyl;

Y is

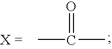

X is Cl⁻; and a pharmaceutically acceptable carrier.

In another embodiment, the invention relates to a method for induction of apoptosis of cancer cells comprising administering to a patient in need thereof a composition comprising an effective amount of a gold(III) complex of formula (III or a pharmaceutically acceptable salt thereof, wherein R₁ and R₄ are absent; R₂ and R₃ together form

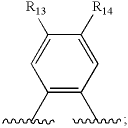

R₅-R₁₂ are each —H;

Y is

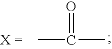

X is Cl⁻; and a pharmaceutically acceptable carrier.

The invention also relates to methods for inhibition of reverse transcriptase of Human Immunodeficiency virus-1.

In one embodiment, the invention relates to a method for inhibition of reverse transcriptase of Human Immunodeficiency virus-1 comprising administering to a patient in need thereof a composition comprising an effective amount of a gold(III) complex of formula (I) or a pharmaceutically acceptable salt thereof, wherein:

R₁, R₄, R₇ and R₁₀ are each independently —H, -halo, —(C₁-C₆)alkyl or —O(C₁-C₆)alkyl, -(6-membered)aryl or -(5 to 10-membered)heteroaryl, each of which may be substituted with one or more -halo, —(C₁-C₆)alkyl, —O(C₁-C₆)alkyl, —OSO₂ or —NO₂; R₂, R₃, R₅, R, 8, R₉, R₁₁ and R₁₂ are each independently —H, —(C₁-C₆)alkyl which may be substituted with one or more —C(O)OR₁₃, -halo or ═O groups;

R₁₃ is —(C₁-C₆)alkyl;

each X^{p} is independently a pharmaceutically acceptable counter-ion;

m is an integer ranging from −3 to 5;

p is an integer ranging from −3 to 3;

n is equal to the absolute value of m/p; and

a pharmaceutically acceptable carrier.

In another embodiment, the invention relates to a method for inhibition of reverse transcriptase of Human Immunodeficiency virus-1 comprising administering to a patient in need thereof a composition comprising an effective amount of a gold(III) complex of formula (I) or a pharmaceutically acceptable salt thereof, wherein R₂, R₃, R₅, R₆, R₈, R₉, R₁₁, and R₁₂ are each —H.; X^{p} is Cl⁻; m is 1; n is 1; and a pharmaceutically acceptable carrier.

In another embodiment, the invention relates to a method for inhibition of reverse transcriptase of Human Immunodeficiency virus-1 comprising administering to a patient in need thereof a composition comprising an effective amount of a gold(III) complex of formula (I) or a pharmaceutically acceptable salt thereof, wherein R₁, R₄, R₇ and R₁₀ are each -phenyl; R₂, R₃, R₅, R₆, R₈, R₉, R₁₁ and R₁₂ are each —H.; X^{p} is Cl⁻; m is 1; n is 1; and a pharmaceutically acceptable carrier.

In another embodiment, the invention relates to a method for inhibition of reverse transcriptase of Human Immunodeficiency virus-I comprising administering to a patient in need thereof a composition comprising an effective amount of a gold(III) complex of formula (I) or a pharmaceutically acceptable salt thereof, wherein R₁, R₄, R₇ and R₁₀ are each -4-methylphenyl; R₂, R₃, R₅, R₆, R₈, R₉, R₁₁ and R₁₂ are each —H.; X^{p} is Cl⁻; m is 1; an is 1; and a pharmaceutically acceptable carrier.

In another embodiment, the invention relates to a method for inhibition of reverse transcriptase of Human immunodeficiency virus-1 comprising administering to a patient in need thereof a composition comprising an effective amount of a gold(III) complex of formula (I) or a pharmaceutically acceptable salt thereof, wherein R₁, R₄, R₇ and R₁₀ are each -4-methoxyphenyl; R₂, R₃, R₅, R₆, R₈, R₉, R₁₁ and R₁₂ are each —H.; X^{p} is Cl⁻; m is 1; n is 1; and a pharmaceutically acceptable carrier.

In another embodiment, the invention relates to a method for inhibition of reverse transcriptase of Human immunodeficiency virus-1 comprising administering to a patient in need thereof a composition comprising an effective amount of a gold(III) complex of formula (I) or a pharmaceutically acceptable salt thereof, wherein R₁, R₄, R₇ and R₁₀ are each -4-bromophenyl; R₂, R₃, R₅, R₆, R₈, R₉, R₁₁ and R₁₂ are each —H.; X^{p} is Cl⁻; m is 1; n is 1; and a pharmaceutically acceptable carrier.

In another embodiment, the invention relates to a method for inhibition of reverse transcriptase of Human Immunodeficiency virus-1 comprising administering to a patient in need thereof a composition comprising an effective amount of a gold(III) complex of formula (I) or a pharmaceutically acceptable salt thereof, wherein R₁, R₄, R₇ and R₁₀ are each -4-chlorophenyl; R₂, R₃, R₅, R₆, R₈, R₉, R₁₁ and R₁₂ are each —H.; X^{p} is Cl⁻; m is 1; n is 1; and a pharmaceutically acceptable carrier.

In another embodiment, the invention relates to a method for inhibition of reverse transcriptase of Human Immunodeficiency virus-I comprising administering to a patient in need thereof a composition comprising an effective amount of a gold(III) complex of formula (I) or a pharmaceutically acceptable salt thereof, wherein R₁, R₄, R₇ and R₁₀ are each -3,4,5-trimethoxyphenyl; R₂, R₃, R₅, R₆, R₈, R₉, R₁₁ and R₁₂ are each —H.; X^{p} is Cl⁻; m is 1; n is 1; and a pharmaceutically acceptable carrier.

In another embodiment, the invention relates to a method for inhibition of reverse transcriptase of Human Immunodeficiency virus-1 comprising administering to a patient in need thereof a composition comprising an effective amount of a gold(III) complex of formula (I) or a pharmaceutically acceptable salt thereof, wherein R₁, R₄, R₇ and R₁₀ are each -3,4,5-trifluorophenyl; R₂, R₃, R₅, R₆, R₈, R₉, R₁₁ and R₁₂ are each —H.; X^{p} is Cl⁻; m is 1; n is 1; and a pharmaceutically acceptable carrier.

In another embodiment, the invention relates to a method for inhibition of reverse transcriptase of Human Immunodeficiency virus-1 comprising administering to a patient in need thereof a composition comprising an effective amount of a gold(III) complex of formula (I) or a pharmaceutically acceptable salt thereof, wherein R₁, R₄, R₇ and R₁₀ are each —H; R₂, R₃, R₅, R₆, R₈, R₉, R₁₁ and R₁₂ are each -ethyl; X^{p} is Cl⁻; m is 1; n is 1; and a pharmaceutically acceptable carrier.

In another embodiment, the invention relates to a method for inhibition of reverse transcriptase of Human Immunodeficiency virus-1 comprising administering to a patient in need thereof a composition comprising an effective amount of a gold(III) complex of formula (I) or a pharmaceutically acceptable salt thereof, wherein R1,R4, R₇ and R10 are each —H; and R₂ and R₁₁ are each -ethyl; R₃, R₅, R₉ and R₁₂ are each -methyl; R₆ and R₈ are each -methyl-3-propanoate; X^{p} is Cl⁻; m is 1; n is 1; and a pharmaceutically acceptable carrier.

In another embodiment, the invention relates to a method for inhibition of reverse transcriptase of Human Immunodeficiency virus-i comprising administering to a patient in need thereof a composition comprising an effective amount of a gold(III) complex of formula (I) or a pharmaceutically acceptable salt thereof, wherein R₁, R₄, R₇ and R₁₀ are each -4-(N-methyl)pyridinium; R₂, R₃, R₅, R₆, R₈, R_{g}, R₁₁ and R₁₂ are each —H; X^{p} is Cl⁻; m is 5; n is 5; and a pharmaceutically acceptable carrier.

In another embodiment, the invention relates to a method for inhibition of reverse transcriptase of Human Immunodeficiency virus-1 comprising administering to a patient in need thereof a composition comprising an effective amount of a gold(III) complex of formula (I) or a pharmaceutically acceptable salt thereof, wherein R₁, R₄, R₇ and R₁₀ are each -4-sulfanatophenyl; R₂, R₃, R₅, R₆, R₈, R₉, R₁₁ and R₁₂ are each —H; X^{p} is Na⁺; m is +3; n is 3; and a pharmaceutically acceptable carrier.

In one embodiment, the invention relates to a method for inhibition of reverse transcriptase of Human Immunodeficiency virus-1 comprising administering to a patient in need thereof a composition comprising an effective amount of a gold(III) complex of formula (II) or a pharmaceutically acceptable salt thereof, wherein:

R₁-R₁₂ are each independently —H, -halo, —(C₁-C₆)alkyl or —O(C₁-C₆)alkyl which may be substituted with one or more —O(C₁-C₆)alkyl or -halo;

X is a counter-anion; and

a pharmaceutically acceptable carrier.

In another embodiment, the invention relates to a method for inhibition of reverse transcriptase of Human Immunodeficiency virus-1 comprising administering to a patient in need thereof a composition comprising an effective amount of a gold(III) complex of formula (II) or a pharmaceutically acceptable salt thereof, wherein R₁-R₄ are each —H; X is Cl⁻; and a pharmaceutically acceptable carrier.

In another embodiment, the invention relates to a method for inhibition of reverse transcriptase of Human Immunodeficiency virus-1 comprising administering to a patient in need thereof a composition comprising an effective amount of a gold(III) complex of formula (II) or a pharmaceutically acceptable salt thereof, wherein R₁-R₁₂ are each —H; X is Cl³¹; and a pharmaceutically acceptable carrier.

In another embodiment, the invention relates to a method for inhibition of reverse transcriptase of Human immunodeficiency virus-1 comprising administering to a patient in need thereof a composition comprising an effective amount of a gold(III) complex of formula (II) or a pharmaceutically acceptable salt thereof, wherein R₃, R₅—R₇ and R₉-R₁₀ are each —H; and R₄ and R₈ are each —Cl; X is Cl⁻; and a pharmaceutically acceptable carrier.

In another embodiment, the invention relates to a method for inhibition of reverse transcriptase of Human immunodeficiency virus-1 comprising administering to a patient in need thereof a composition comprising an effective amount of a gold(III) complex of formula (II) or a pharmaceutically acceptable salt thereof, wherein R₁-R₅, R₇ R₉, R₁₁ are each —H; and R₆, R₈, R₁₀ and R₁₂ are each —Cl; X is CF; and a pharmaceutically acceptable carrier.

In one embodiment, the invention relates to a method for inhibition of reverse transcriptase of Human Immunodeficiency virus-1 comprising administering to a patient in need thereof a composition comprising an effective amount of a gold(III) complex of formula (III) or a pharmaceutically acceptable salt thereof, wherein:

(a) R₁-R₁₂ are each independently —H, -halo, —(C₁-C₆)alkyl —O(C₆)alkyl which may be substituted with one or more —O(C₁-C₆)alkyl or -halo; or

(b) R₁ and R₄ are absent; and R₂ and R₃ together form a 6-membered aryl ring of formula

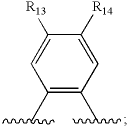

Y is

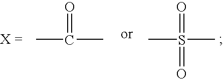

R₁₃ and R₁₄ are each —H or -halo;

X is a counter-anion; and

a pharmaceutically acceptable carrier.

In another embodiment, the invention relates to a method for inhibition of reverse transcriptase of Human Immunodeficiency virus-1 comprising administering to a patient in need thereof a composition comprising an effective amount of a gold(III) complex of formula (III or a pharmaceutically acceptable salt thereof, wherein

Y is

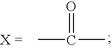

X is Cl⁻; and a pharmaceutically acceptable carrier.

In another embodiment, the invention relates to a method for inhibition of reverse transcriptase of Human Immunodeficiency virus-1 comprising administering to a patient in need thereof a composition comprising an effective amount of a gold(III) complex of formula (III or a pharmaceutically acceptable salt thereof, wherein R₁-R₁₂ are each —H;

Y is

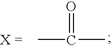

X is Cl⁻; and a pharmaceutically acceptable carrier.

In another embodiment, the invention relates to a method for inhibition of reverse transcriptase of Human immunodeficiency virus-1 comprising administering to a patient in need thereof a composition comprising an effective amount of a gold(III) complex of formula (III or a pharmaceutically acceptable salt thereof, wherein R₁-R₄ are each -methyl; R₅-R₁₂ are each —H;

Y is

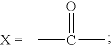

X is Cl⁻; and a pharmaceutically acceptable carrier.

In another embodiment, the invention relates to a method for inhibition of reverse transcriptase of Human Immunodeficiency virus-1 comprising administering to a patient in need thereof a composition comprising an effective amount of a gold(III) complex of formula (III or a pharmaceutically acceptable salt thereof, wherein R₁ and R₄-R₁₂ are each —H; R₂ and R₃ are each -phenyl;

Y is

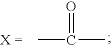

X is Cl⁻; and a pharmaceutically acceptable carrier.)

In another embodiment, the invention relates to a method for inhibition of reverse transcriptase of Human immunodeficiency virus-I comprising administering to a patient in need thereof a composition comprising an effective amount of a gold(III) complex of formula (III or a pharmaceutically acceptable salt thereof, wherein R1 and R4 are absent; R₂ and R₃ together form

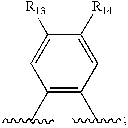

R₅—R₁₂ are each —H;

Y is

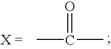

X is Cl⁻; and a pharmaceutically acceptable carrier.

The invention also relates to pharmaceutical compositions comprising a Gold(III) Complex and a pharmaceutically acceptable carrier.

In one embodiment, the invention relates to a composition comprising an effective amount of a gold(III) complex of formula (I) or a pharmaceutically acceptable salt thereof, wherein:

R₁, R₄, R₇ and R₁₀ are each independently —H, -halo, —(C₁-C₆)alkyl or —O(C₁-C₆)alkyl, -(6-membered)aryl or -(5 to 10-membered)heteroaryl, each of which may be substituted with one or more -halo, —(C₁-C₆)alkyl, —O(C₁-C₆)alkyl, —OSO₂ or —NO₂; R₂, R₃, R₅, R₆, R₈, R₉, R₁₁ and R₁₂ are each independently —H, —(C₁-C₆)alkyl which may be substituted with one or more —C(O)OR₁₃, -halo or ═O groups;

R₁₃ is —(C₁-C₆)alkyl;

each X^{p} is independently a pharmaceutically acceptable counter-ion;

m is an integer ranging from −3 to 5;

p is an integer ranging from −3 to 3;

n is equal to the absolute value of m/p; and

a pharmaceutically acceptable carrier.

In one embodiment, the invention relates to a composition comprising an effective amount of a gold(III) complex of formula (II) or a pharmaceutically acceptable salt thereof, wherein:

R₁-R₁₂ are each independently —H, -halo, —(C₁-C₆)alkyl or —O(C₁-C₆)alkyl which may be substituted with one or more —O(C₁-C₆)alkyl or -halo;

X is a counter-anion; and

a pharmaceutically acceptable carrier.

In one embodiment, the invention relates to a composition comprising an effective amount of a gold(III) complex of formula (III) or a pharmaceutically acceptable salt thereof, wherein:

(a) R₁-R₁₂ are each independently —H, -halo, —(C₁-C₆)alkyl —O(C₆)alkyl which may be substituted with one or more —O(C₁-C₆)alkyl or -halo; or

(b) R₁ and R₄ are absent; and R₂ and R₃ together form a 6-membered aryl ring of formula

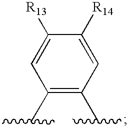

Y is

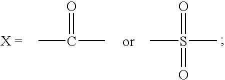

R₁₃ and R₁₄ are each —H or -halo;

X is a counter-anion; and

a pharmaceutically acceptable carrier.

Scheme 2 shows illustrative examples of gold(III) porphyrin complexes useful in the present invention, where the counter-ion for Complexes 1a-1j is chloride, and the counter-cation for Complex 1k is Na⁺.

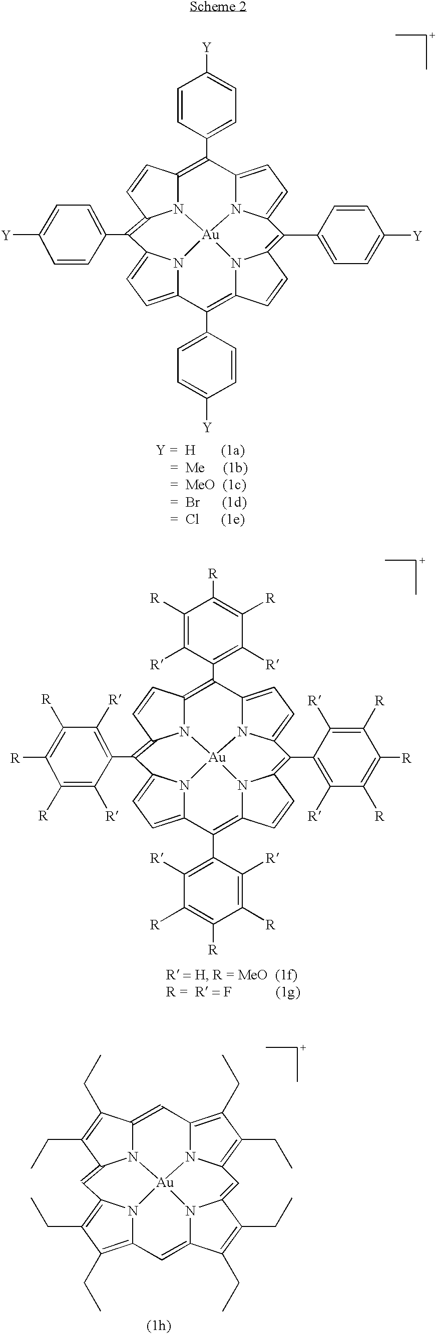

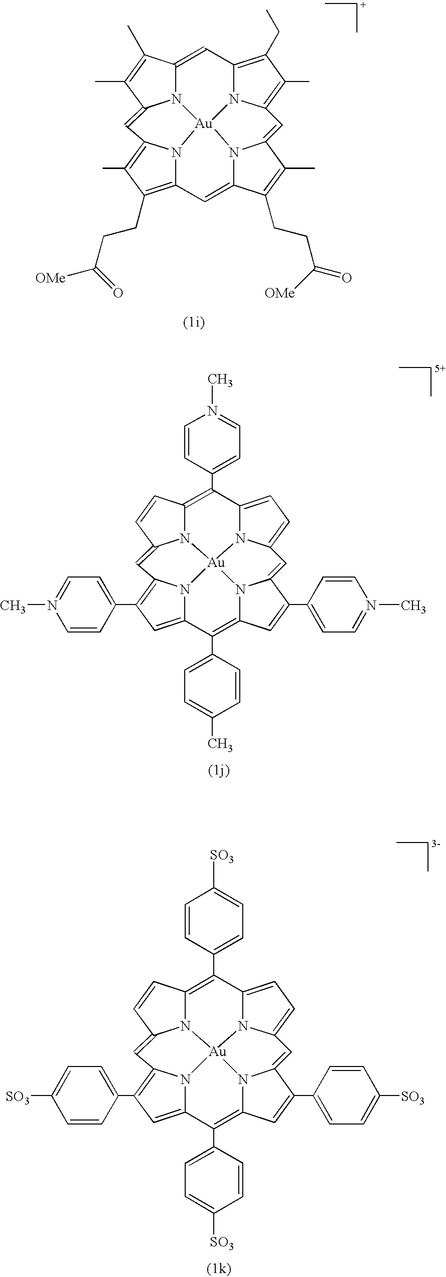

Scheme 3 shows illustrative non-limiting examples of gold(III) Shiff base complexes (2) and gold(III) bis(pyridyl)carboxamides complexes (3) useful in the present invention, where the counter-anion is Cl⁻.

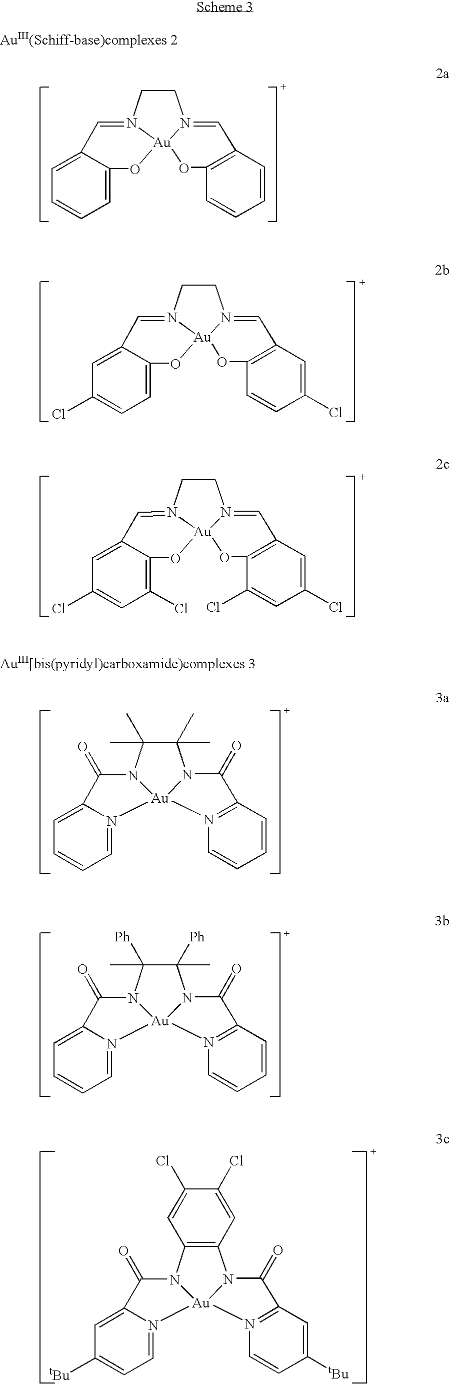

The Complexes 1a-i (Scheme 2) can be prepared according to literature methods with some modifications (see MacCragh et al., J. Am. Chem. Soc. 87:2496 (1965) and Fleischer et al., Inorg. Nucl. Chem. Lett. 5:373-376 (1969)) by treating KAuCl₄ or nBu₄NAuCl₄ with the free base porphyrins in the presence of NaOAc in acetic acid. The reaction are performed under a nitrogen atmosphere using standard Schlenk technique. Complexes 1a-i (Scheme 2) were This process is depicted in Scheme 4, Route 1.

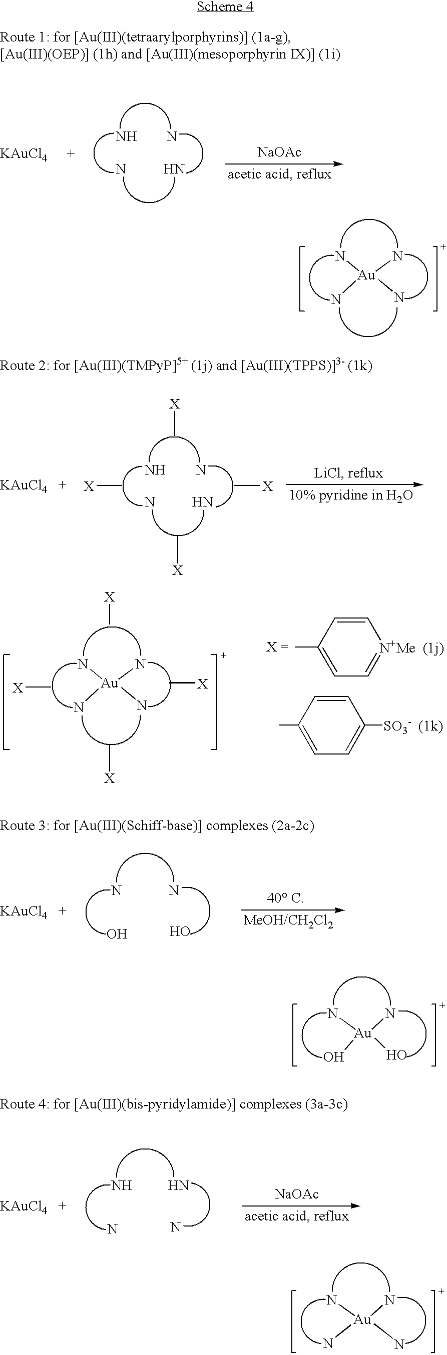

After purification with column chromatography using alumina, followed by metathesis with LiCl in aqueous acetone, analytically pure gold(III) porphyrin complexes were obtained as a chloride salt in 60-70% yields (see Examples Section).

Complexes 1j and 1k can be prepared according to published procedures in a manner similar to that used to prepare Complexes 1a-1i except that the reaction was performed using 10% aqueous pyridine as the solvent (see Jamin et al., Inorg. Chim. Acta 27:135-143 (1978)); Gibbs et al., J. Inorg. Biochem. 32:39-65 (1988); and Pasternack, J. Inorg. Nucl. Chem. 36:599 (1974)) to afford Complexes 1j and 1k in about 35% yield. This procedure is depicted in Scheme 4, Route 2.

The gold(III) Schiff base Complexes 2a-2c can be prepared in a manner similar to that reported by Barnholtz et al., Inorg. Chem. 40:972 (2001). Treatment of free Schiff-base ligand (0.15 mmol) and KAuCl₄ (0.037 mmol) in a CH₂Cl₂/MeOH mixture (5:1) at room temperature would afford the gold(III) Schiff-base complexes in about 25% isolated yields. This procedure is depicted in Scheme 4, Rout 4.

The gold(III)bis(pyridyl)carboxamide Complexes 3a-3c can be prepared in a manner similar to that described above for Complexes 2a-2c by reaction of KAuCl₄ with the free base bis(pyridyl)carboxamides in the presence of NaOAc in acetic acid. After extensive washing with distilled water and metathesis with LiCl in aqueous acetone, analytically pure gold(III) bis(pyridyl)carboxamide complexes are obtained as chloride salts in 50-60% yields. This procedures is depicted Scheme 4, Routes 4.

Meso-tetraphenylporphyrin (H₂TPP), meso-tetrakis(4-tolyl)porphyrin (H₂TTP), meso-tetrakis(4-methoxyphenyl)porphyrin (H₂TOMePP), meso-tetrakis(4-bromophenyl)porphyrin (H₂TBP), meso-tetrakis(4-chlorophenyl)porphyrin (H₂TCP) and meso-tetrakis(3,4,5-trimethoxyphenyl)porphyrin (H₂TTMPP) can be prepared according to literature methods or obtained from commercial sources. See Adler, J. Org. Chem. 32:476 (1967); Keinan et al., Inorg. Chem. 31:5433-5438 (1992); and Barnett et al., Tetrahed. Lett. 30:2887-2888 (1973). Meso-tetrakis(N-methylpyridinium-4-yl)porphyrin (H₂TMPyP), 2,3,7,8,12,13,17,18-octaethylporphyrin (H₂OEP)) and mesoporphyrin IX (H₂ MP) are available from Aldrich Chemical, Milwaukee, Wis. Meso-tetrakis(pentafluorophenyl)porphyrin (H₂TF5PP) and meso-tetrakis(4-sulfonatophenyl)porphyrin (H₂TPPS) are available from Fluka Chemical (Buchs, Switzerland).

Tetrabutylammonium tetrachloroaurate (n-Bu₄NAuCl₄) can be prepared from the metathesis reaction of [nBu₄N]Cl with HAuCl₄ in 0.01 M HCl.

In particular, the present invention relates to the use of gold(III) porphyrin complexes and related analogues containing a tetradentate dianionic macrocyclic ligand (see Schemes 2 and 3, class III, for examples) as a new class of anti-tumor gold(III) compounds.

Due to high redox potential, gold(III) complexes are usually unstable in solution and readily undergo decomposition to gold(I)/colloidal gold in buffer solution. However, the Gold(III) Complexes of the present invention are stable under physiologically relevant conditions. For example, without being limited by theory, stability of the gold(III) porphyrin complexes 1a-1k compounds is believed to be due in part to: (1) strong σ-donors to stabilize oxidizing metal centers, (2) strong chelating effect to avoid undesirable demetallation, and (3) rigid ligand scaffold (especially porphyrin) to stabilize the four-coordinated gold(III) center by raising the kinetic barrier (inner-sphere reorganization energy) for reduction to a two-coordinate gold(I) center.

In some embodiments, the Gold(III) Complexes may coordinated to another molecule, e.g., a ligand. Molecules or ligands that can coordinate to a Gold(III) Complex include, but are not limited to, porphyrins, metalloporphyrins, amino acids, peptides, polypeptides, proteins, nucleotides, polynucleotides, DNA, RNA, donor and acceptor groups, antigens, antibodies antiviral compounds and anticancer compounds. It will be understood that when the Gold(III) Complexes are coordinated to an oligomeric or polymeric molecule, such molecule may be linear or branched.

In one embodiment, the ligand is selected from the group consisting of porphyrins, metalloporphyrins, amino acids, peptides, polypeptides, proteins, nucleotides, polynucleotides, deoxyribonucleic acid, and ribonucleic acid.

Non-limiting examples of porphyrin or porphyrin-like complexes include porphyrins isolated from nature; synthetic porphyrins; phthalocyanines; chlorins; substituted porphyrins or porphyrin-like compounds having symmetrically and unsymmetrically located substituents on any of the positions of the ring periphery; neutrally charged porphyrins; positively charged porphyrins; negatively charged porphyrins; charged porphyrins in combination with counter-ions including alkaline, alkaline earth metal; and rare-earth metal ions.

Non-limiting examples of antiviral compounds include HPA-23, interferons, ribavirin, phosphonoformate, ansamycin, suramin, imuthiol, pencillamine, carbovir, 3′-azido-3′-deoxythymidine, 2′,3′-dideoxycytidine, 2′,3′-dideoxyinosine, 2′,3′-dideoxyadenosine, 3′-azido-2′,3′-dideoxyuridine, 2′,3′-dideoxy-2′,3′-didehydrocytidine, 3′-deoxy-2′,3′-didehydrothymidine and 3′-azido-5-ethyl-2′,3′-dideoxyuridine.

Non-limiting examples of anticancer compounds cisplatin, carboplatin, bleomycin, vincristine, vinblastine, doxorubicin, cyclophosphamide, prednisone, methotrexate, dexamethasone, leucovorin and blenoxane.

Non-limiting examples of cancer cells include mammalian carcinoma cells; and cultured cancer such as Human Cervix Epitheloid Carcinoma cells, Oral Epidermoid carcinoma cells, Nasopharyngeal carcinoma cells, Human Promyelocytic Leukemia cells and Human Hepatocellular Carcinoma cells.

The Gold(III) Complexes are useful for the induction of apoptosis of cancer cells. Cell deaths can be classified into two types, necrosis (“accidental” cell death) and apoptosis (“programmed” cell death). Necrosis causes severe inflammation, but apoptosis does not. Harmlessly disposing of cells (e.g. cancer cells) is one of the considerations in chemotherapy. Therefore, induction of apoptosis is one of the considerations in anti-cancer drug development. Most of the cytotoxic anti-cancer drugs in current clinical uses have been shown to induce apoptosis in susceptible cells. Applicants in vitro studies show that that the Gold(III) Complexes are useful for the induction of apoptosis of cancer cells. Without being limited by theory, it is believed that the Gold(III) Complexes “nick” cellular DNA and cause oxidative stress.

In particular, DNA is one of the major targets for anticancer drugs. See Hurley et al., Nature Reviews Cancer, 2:188-200 (2002). It is well known that porphyrins and metalloporphyrins would interact with DNA by intercalation and minor groove binding. Earlier works by Liu and Lippard showed that gold(III) complexes containing polypyridine ligands such as (C\hat{ }N\hat{ }N) and terpyridine (terpy) bind to DNA by intercalation. See Liu et al., J. Chem. Soc., Chem. Comm. 17:1787-1788 (1995); and Hollis et al., J. Am. Chem. Soc. 105:4293-4299 (1983)]. According to the work by Ward and Dabrowiak, Et₃PAuBr₃ would covalently bind to DNA through N7 of guanine (see Ward et al., J. Am. Chem. Soc. 1987, 109:3810-3811 (1987). In conjunction to the observed cytotoxicity of the gold(III) porphyrins, we have investigated the binding interaction of Complex 1a with DNA.

Reverse transcriptase represents one of the major targets in the development of chemotherapeutic drugs against HIV. The Gold(III) Complexes are also useful inhibition of reverse transcriptase. In one embodiment, the reverse transcriptase is the reverse transcriptase of Human Immunodeficiency virus-1.

In some embodiments, the Gold(III) Complexes of the invention may be used in combination with at least one other Gold(II) Complex or another therapeutic agent. Non-limiting examples of other therapeutic agents include anti-cancer agents and anti-HIV agents. In one embodiment, the anti-HIV agent is AZT.

In one embodiment, the invention relates to a method for inhibition of reverse transcriptase of Human Immunodeficiency virus-1 comprising administering to a patient in need thereof a composition comprising an effective amount of a gold(III) complex of formula (I) or a pharmaceutically acceptable salt thereof, wherein:

R₁, R₄, R₇ and R₁₀ are each independently —H, -halo, —(C₁-C₆)alkyl or —O(C₁-C₆)alkyl, -(6-membered)aryl or -(5 to 10-membered)heteroaryl, each of which may be substituted with one or more -halo, —(C₁-C₆)alkyl, —O(C₁-C₆)alkyl, —OSO₂ or —NO₂;

R₂, R₃, R₅, R₆, R₈, R₉, R₁₁ and R₁₂ are each independently —H, —(C₁-C₆)alkyl which may be substituted with one or more —C(O)OR₁₃, -halo or ═O groups;

R₁₃ is —(C₁-C₆)alkyl;

each X^{p} is independently a pharmaceutically acceptable counter-ion;

m is an integer ranging from −3 to 5;

p is an integer ranging from −3 to 3;

n is equal to the absolute value of m/p;

AZT; and

a pharmaceutically acceptable carrier.

In another embodiment, the invention relates to a method for inhibition of reverse transcriptase of Human Immunodeficiency virus-1 comprising administering to a patient in need thereof a composition comprising an effective amount of a gold(III) complex of formula (I) or a pharmaceutically acceptable salt thereof, wherein R₁, R₄, R₇ and R₁₀ are each -4-(N-methyl)pyridinium; R₂, R₃, R₅, R₆, R₈, R₉, R₁₁ and R₁₂ are each —H; X^{p} is Cl⁻; m is 5; n is 5; AZT; and a pharmaceutically acceptable carrier.

The following examples are set forth to assist in understanding the invention and should not be construed as specifically limiting the invention described and claimed herein. Such variations of the invention, including the substitution of all equivalents now known or later developed, which would be within the purview of those skilled in the art, and changes in formulations or minor changes in experimental design, fall within the scope of the present invention.

### EXAMPLES

Materials. All chemicals except specially mentioned were purchased from Sigma-Aldrich Chemical Co.(Tokyo, Japan). Acetonitrile and dichloromethane were freshly distilled from CaH₂. Analytical grade organic solvents and double distilled deionized water were used throughout the experiments.

The calf thymus DNA was purchased from Sigma-Aldrich Chemical Co. and was purified by phenol-chloroform extraction. The DNA was dissolved in buffer (5 mM Tris, 5 mM NaCl, pH 7.2). The concentration (per base pair) of calf thymus DNA was determined spectrophotometrically on the basic of ε₂₆₀, 13,200 M⁻¹cm⁻¹bp.

Human promyelocytic leukemia (HL-60), human hepatocellular carcinoma (HepG2), Human cervix epitheloid carcinoma cells (HeLa) and human oral epidermoid carcinoma cells (KB-3-1), were obtained from American Type Culture Collection (ATCC) (Rockville, Md.). The multi-drug-resistant variant KB-V1 cell line was obtained by stepwise selections for vinblastine resistance as described by Shen et al., J. Biol. Chem. 261:7762-7770 (1986)], and was generously provided by Dr Michael Gottesman (NIH, Bethesda, Md.). Human nasopharyngeal carcinoma cells (SUNE1 and its cisplatin resistance variant, CNE1) were derived from poorly differentiated NPC in Chinese patients as described by Gu et al., Chin. J. Cancer 2:270-272 (1983), and were generously provided by Dr S. W. Tsao (The University of Hong Kong, Hong Kong, PRC). Cell culture flasks and 96 well microtitre plates were from Nalge Nunc hit. Culture medium, other medium constituents and phosphate buffered saline (PBS) were obtained from Gibco BRL (Rockville, Md.). Cell Proliferation Kit I (MTT) was purchased from Roche (Mannheim, Germany).

Instrumentation. All absorption spectra were recorded on a Perkin-Elmer Lambda 19 or a Varian Cary 50 UV-visible spectrophotometer equipped with a PCB-150 water circulator. ¹H NMR spectra were recorded on Bruker DPX-300 or DPX-500 NMR spectrometers. Positive ion FAB mass spectra were recorded on a Finnigan MAT95 mass spectrometer. Viscosity evaluation was carried out by using a Cannon-Manning Semi-Micro Viscometer (Cannon-Instrument Co., State College, Pa.). Cyclic voltammetry was performed by using a PAAR model 175 universal programmer and a Model 173 potentiostat, and the cyclic voltammograms were recorded with a Kipp & Zonen BD 90×Y recorder at scan rates of 200 mVs⁻¹. Flow cytometric analysis was performed with a Coulter EPICS flow cytometer (Coulter, Miami, Fla.) equipped with 480 long, 525 band and 625 long pass mirrors. Samples were excited by 15 mW air-cool argon convergent laser at 488 nm. Fluorescence signals were manipulated with Coulter Elite 4.0 software (Coulter) and were analyzed by Winlist 1.04 and Modfit 5.11 software (Verity Software House, Topsham, Me.).

All the complexes were spectroscopically characterized by UV-vis, ¹H NMR and FAB-MS with purities of >99% being confirmed by elemental analysis.

### EXAMPLE 1

### Preparation of the Gold(III) Complexes

Example 1 describe the preparation and characterization of illustrative Gold(III) Complexes 1a-1k.

Gold(III) Complexes 1a-1i. The synthesis of the gold(III) porphyrins was performed under a nitrogen atmosphere using the standard Schlenk technique. Complexes 1a-i (Scheme 2) were prepared according to a literature method with some modifications. See MacCragh et al., J. Am. Chem. Soc. 87:2496 (1965) and Fleischer et al., Inorg. Nucl. Chem. Lett. 5:373-376 (1969). In general, KAuCl₄ or [nBu₄N]AuCl₄ (0.508 mmol) and sodium acetate (2.538 mmol) were heated to 80° C. in acetic acid (20 ml) for 15 minutes. A solution of free porphyrin (0.406 mmol) in acetic acid (10 ml) was added dropwise. The mixture was heated under reflux for 0.5 to 2 h, and the completion of metallation was checked by disappearance of the Q band using UV-Vis spectrophotometry. Upon removal of solvent under vacuum, the residue was dissolved in CH₂Cl₂ (40 ml). The CH₂Cl₂ solution was washed twice with water (2×40 ml) to remove any unreacted KAuCl₄ and NaOAc, and pre-concentrated down to 3 ml. It was chromatographed on a neutral 90-alumina packed column. The column was eluted with CH₂Cl₂ to remove the unreacted free base porphyrin, and the gold(III) porphyrin complex was eluted using CH₂Cl₂/MeOH (99:1, v/v). A reddish-purple solid was obtained after solvent evaporation and the complex was recrystallized from a CHCl₃/petroleum ether (1:1, v/v) mixture.

Gold(III) Complexes 1j-k. Complexes 1k and 1k were prepared according to published procedures. See Gibbs et al., J. Inorg. Biochem. 32:39-65 (1988); and Pasternack, J. Inorg. Nucl. Chem., 36:599 (1974).

Gold(III) Complexes 1a-1k exhibited well-resolved ¹H NMR spectra. For complexes 1a-1g and 1j-1k, all the pyrrolic hydrogen atoms are equivalent and are consistent with a D₄ symmetry.

Analytical data for Complexes 1a-1k are shown below:

Complex 1a: UV-vis (CH₂Cl₂) λₘₐₓ/nm (log ε): 409 (5.68), 521 (4.73). ¹H NMR (CDCl₃): δ 9.28 (s, 8H), 7.89 (m, 8H), 8.24 (d, 8H) 7.89 (m, 4H). m/z=809. Yield 70%. Anal. Calcd. for C₄₄H₂₈N₄C1Au (%): C, 62.53; H, 3.34; N, 6.63. Found: C, 62.50; H, 3.61; N, 6.74.

Complex 1b: UV-vis (CH₂Cl₂) λₘₐₓ/nm (log F): 413 (5.41), 522 (4.62). ¹H NMR (CDCl₃): δ 9.29 (s, 8H), 7.67 (d, 8H), 8.10 (d, 8H) 2.75 (s, 12H). m/z=865. Yield 69%. Anal. Calcd. for C₄₈H₃₆N₄ClAu (%): C, 63.97; H, 4.03; N, 6.22. Found: C, 63.82; H, 4.39; N, 5.82.

Complex 1c: UV-vis (CH₂Cl₂) λₘₐₓ/nm (log ε): 423 (5.26), 527 (4.34). ¹H NMR (CDCl₃): δ 9.31 (s, 8H), 7.38 (d, 8H), 8.13 (d, 8H) 4.13 (s, 12H). m/z=929. Yield 63%. Anal. Calcd for C₄₈H₃₆N₄O₄ClAu (%): C, 59.73; H, 3.76; N, 5.80. Found: C, 59.55; H, 3.49; N, 5.99.

Complex 1d: UV-vis (CH₂Cl₂) λₘₐₓ/nm (log ε): 411 (5.53), 522 (4.48). ¹H NMR (CDCl₃): δ 9.24 (s, 8H), 7.80 (d, 8H), 8.14 (d, 8H). m/z=1125. Yield 60%. Anal. Calcd. for C₄₄H₂₄N₄Br₄ClAu (%): C, 45.53; H, 2.08; N, 4.83. Found: C, 46.01; H, 2.35; N, 4.79.

Complex 1e: UV-vis (CH₂Cl₂) λₘₐₓ/nm (log ε): 409 (5.47), 520 (4.34). ¹H NMR (CDCl₃): δ 9.23 (s, 8H), 7.88 (d, 8H), 8.20 (d, 8H). m/z=947. Yield 61%. Anal. Calcd. for C₄₄H₂₄N₄Cl₅Au (%): C, 53.77; H, 2.46; N, 5.70. Found: C, 53.46; H, 2.72 N, 5.68.

Complex 1f: UV-vis (CH₂Cl₂) λₘₐₓ/nm (log ε): 423 (5.18), 526 (4.17). ¹H NMR (CDCl₃): δ 9.36 (s, 8H), 7.57 (s, 8H), 3.98 (s, 24H) 4.19 (s, 12H). m/z=1169. Yield 70%. Anal. Calcd. for C₅₆H₅₂O₁₂N₄ClAu (%): C, 55.80; H, 4.35; N, 4.65. Found: C, 55.56; H, 4.28; N, 4.57.

Complex 1 g: UV-vis (CH₂Cl₂) λₘₐₓ/nm (log F): 402 (5.71), 518 (4.55), 552 (4.10). ¹H NMR (CDCl₃): δ 9.48 (s, 8H). m/z=1170. Yield 60%. Anal. Calcd. for C₄₄H₈N₄F₂₀ClAu: C, 43.86; H, 0.67; N, 4.65. Found: C, 43.93; H, 0.65; N, 4.62.

Complex 1 h: UV-vis (CH₂Cl₂) λₘₐₓ/nm (log ε): 389 (5.35), 510 (3.98), 545 (4.23). ¹H-NMR (CDCl₃): δ 10.51 (s, 8H), 2.10 (t, 24H), 1.59 (q, 16H) 7.89 (m, 4H). m/z=731. Yield 62%. Anal. Calcd. for C₃₆H₄₄N₄ClAu (%): C, 56.51; H, 5.80; N, 7.32. Found: C, 56.14; H, 5.67; N, 7.58.

Complex 1i: UV-vis (CH₂Cl₂) λₘₐₓ/nm (log ε): 398 (5.12), 518 (3.89), 548 (4.12). ¹H NMR (CDCl₃): δ 10.63 (s, 4H). m/z=789. Yield 64%. Anal. Calcd. for C₃₆H₄₂O₄N₄ClAu (%): C, 52.27; H, 5.12; N, 6.77. Found: C, 51.94; H, 4.85; N, 6.57.

Complex 1j: UV-vis (H₂O) λₘₐₓ/nm (log ε): 405 (4.89), 522 (3.98), 556 (3.64). ¹H NMR (D₂O): δ 9.52 (s, 8H), 9.33 (d, 8H), 9.03 (d, 8H) 2.07 (s, 12H). m/z=175. Yield 34%. Anal. Calcd. for C₄₄H₆₀N₈O₁₂Cl₅Au (%): C, 41.70; H, 4.77; N, 8.84. Found: C, 41.98; H, 4.65; N, 8.57.

Complex 1k: UV-vis (H₂O) λₘₐₓ/nm (log ε): 407 (5.34), 521 (4.39). ¹H NMR (D₂O): δ 8.62 (s, 8H), 7.69 (d, 8H), 8.24 (d, 8H). m/z=375. Yield 42%. Anal. Calcd. for C₄₄H₄₀N₄O₂₀ClS₄Na₄Au (%): C, 37.82; H, 2.89; N, 4.01. Found: C, 37.53; H, 3.01; N, 4.17.

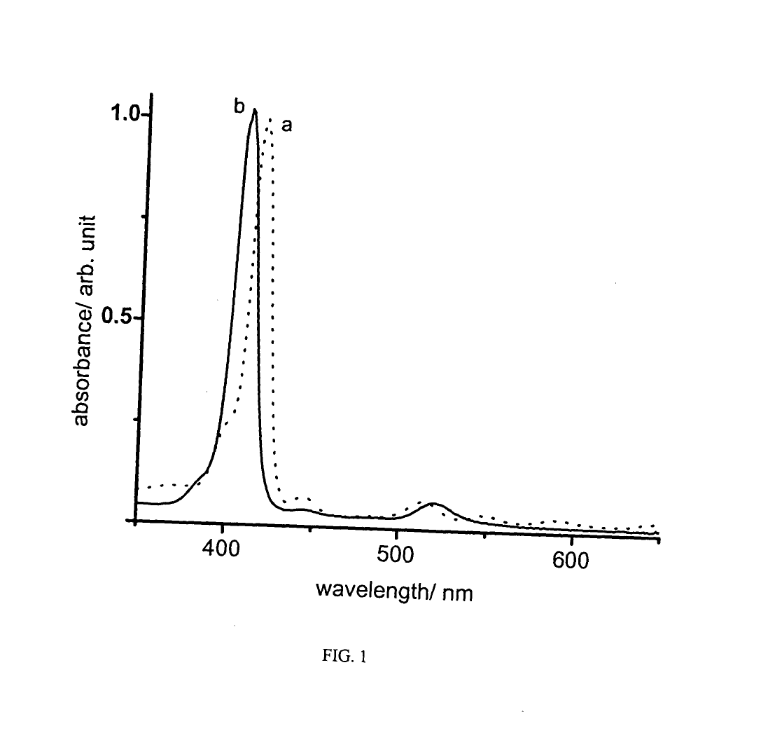

The UV-vis spectra of Complexes 1a-1k (the gold porphyrin complexes) feature an intense Soret band and a weaker Q band. Compared to the spectrum of a closed d-shell metalloporphyrin, the Soret bands of the metal complexes of the invention display a characteristic blue-shift in absorption energy (hypso, see FIG. 1). Without being bound by theory, it is believed that the Goutermann's four-orbital-model, the hypso spectrum for gold(III) porphyrins is the result of metal-to-ligand π-back bonding interaction between the metal filled d_{π} and the vacant π* orbital of the porphyrin ligand.

X-ray Crystal Determination. Crystals of Complex 1a were obtained by a slow diffusion of n-pentane into a solution of Complex 1a in chloroform. A purple crystal having dimensions 0.4×0.1×0.1 mm mounted on a glass fiber was used for data collection at 28° C. on a MAR diffractometer with a 300 mm image plate detector using graphite monochromatized Mo-K_{α} radiation (λ=0.71073 Å). Data collection was made with 3° oscillation step of (p, 300 seconds exposure time and scanner distance at 120 mm. 48 images were collected. The images were interpreted and intensities integrated using program DENZO [Otwinowski, Z. and Minor, W. In Processing of X-ray Diffraction Data Collected in Oscillation Mode, Methods in Enzymology, Carter C. W., Sweet Jr. & R. M., Eds.; Academic Press.: 1997; Vol. 276, p. 307-326]. The structure was solved by direct methods employing SHELXS-97 program [Sheldrick, G., M. SHELX97. Programs for Crystal Structure Analysis (Release 97-2). University of Goetingen, Germany, 1997]. Au, Cl and many non-H atoms were located according to the direct methods. The positions of the other non-hydrogen atoms were found after successful refinement by full-matrix least-squares using program SHELXL-97.

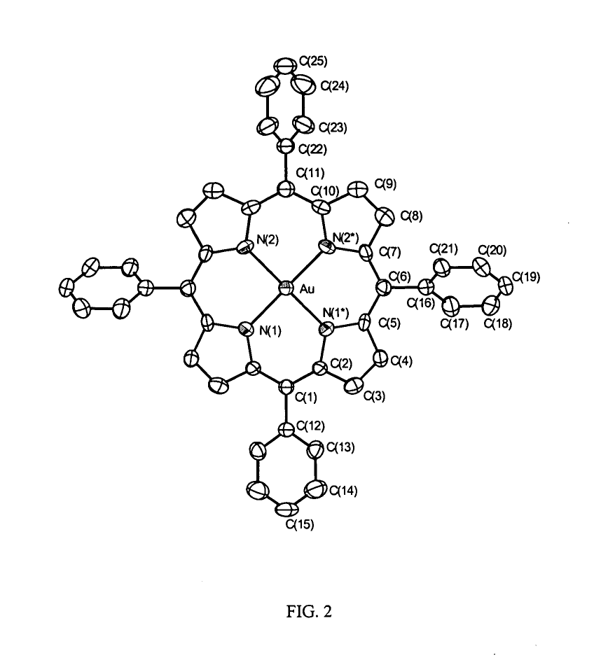

The molecular structure of Complex 1a was established by X-ray crystallography (FIG. 2). Crystallographic data collection parameters and selected bond angles and distances for Complex 1a are summarized in Table 1 and Table 2, respectively. As shown in FIG. 2, the gold(III) center is four-coordinated and is located within the plane of the tetrapyrrole macrocycle. The Au—N distances are 2.032(5) and 2.033(5) Å, which are comparable to the related distances (1.93 to 2.14 Å) found in the literature. See, e.g., Hollis et al., J. Am. Chem. Soc. 105:4293-4299 (1983), which discloses [Au(terpy)Cl]²⁺ (terpy=2,2′,2″-terpyridine); Liu et al., J. Chem. Soc., Chem. Comm. 17 :1787-1788 (1995), which discloses [Au(C\hat{ }N\hat{ }N)Cl]⁺ (C\hat{ }N\hat{ }N=6′-phenyl-2,2′-bipyridine); and Calamai et al., Inorg. Chim. Acta 285:309-312 (1999), which discloses [Au(esal)]²⁺ (esal=N-ethylsalicylaldiminate).

TABLE 1
X-ray Crystallographic Data for Complex 1a.

| | | |
|---|---|---|
| empirical formula | C₄₄H₂₈AuClN₄O₄ | |
| formula weight | 909.12 | |
| temperature | 253(2) K | |
| wavelength | 0.71073 Å | |
| crystal system | Orthorhombic | |
| space group | P n n a | |
| unit cell dimensions | a = 8.1020(16) Å | α = 90° |
| | b = 20.964(4) Å | β = 90° |
| | c = 20.060(4) Å | γ = 90° |
| volume | 3407.2(12) Å | |
| Z | 4 | |
| density (calculated) | 1.772 mg/m³ | |
| absorption coefficient | 4.451 mm⁻¹ | |
| F(000) | 1792 | |
| crystal size | 0.4 × 0.1 × 0.1 mm³ | |
| theta range for data collection | 1.40 to 25.63° | |
| index ranges | −9 <= h <= 9, −24 <= k <= 25, −24 <= 1 <= 24 | |
| reflections collected | 13426 | |
| independent reflections | 3006 [R(int) = 0.0478] | |
| completeness to theta = 25.63° | 95.3% | |
| absorption correction | None | |
| refinement method | Full-matrix least-squares on F² | |
| data/restraints/parameters | 3066/0/248 | |
| goodness-of-fit on F² | 0.934 | |
| final R indices [I > 2sigma(I)] | R₁ = 0.0308, wR₂ = 0.0790 | |
| R indices (all data) | R₁ = 0.0645, wR₂ = 0.1040 | |
| largest diff, peak and hole | 0.716 and −1.590 e Å⁻³ | |

TABLE 2
Selected Bond Distances (Å) and Bond Angles (deg) for Complex 1a.

| | Bond Distances (Å) |
|---|---|
| Au—N(1) | 2.032(5) |
| Au—N(1*) | 2.032(5) |
| Au—N(2*) | 2.033(5) |
| Au—N(2) | 2.033(5) |
| | Bond Angles (deg) |
| N(1)-Au—N(1*) | 89.8(3) |
| N(1)-Au—N(2*) | 177.31(19) |
| N(1*)-Au—N(2*) | 90.1(2) |
| N(1)-Au—N(2) | 90.1(2) |
| N(1*)-Au—N(2) | 177.31(19) |
| N(2*)-Au—N(2) | 90.1(3) |

Gold(III) Complexes 2 (Schiff base) and 3c (Bis(pyridyl)carboxamide): Gold(III) Complexes 2a and 3c were prepared by reacting K[Au^{III}Cl₄] with the corresponding ligands in refluxing acetonitrile (for 2a) and acetic acid (for 3c) according to the reported procedures (see Barnholtz et al, Inorg. Chem. 40: 972-976, (2001)). The complexes were characterized by ¹H-NMR and FAB-MS spectroscopies.

Complex 2a: FAB-MS: m/z=463 (M⁺).

Complex 3c: FAB-MS: m/z=694 (M)⁺].

### EXAMPLE 2

### Stablity Studies of the Gold(III) Complexes

Example 2 describes the results of stability studies for illustrative Gold(III) Complexes 1a, 1b, 1c, 1d, 1e, 2a and 3c.

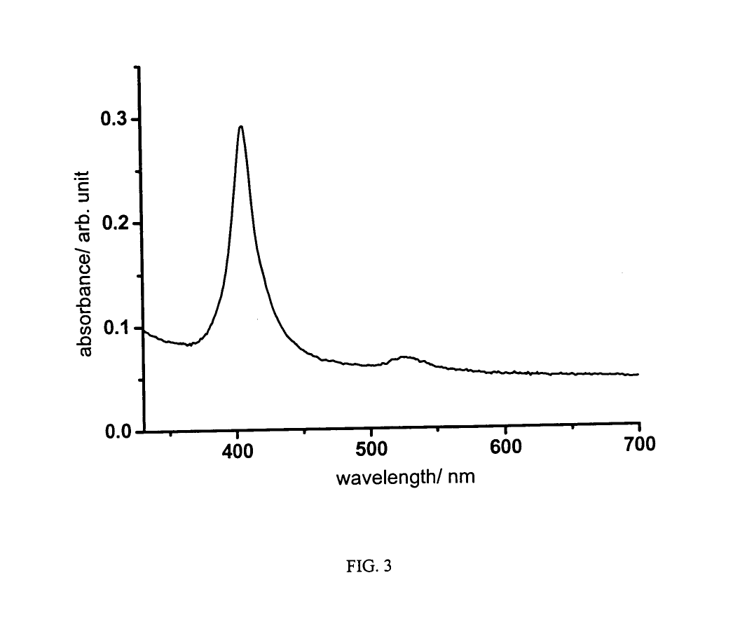

UV-vis experiments. For UV-vis analysis, the absorption spectra of 2.5 μM (7 nmoles) of all studied complexes in (1:19) MeCN/Tris buffer solution was monitored over 72 h. A solution of Tris buffer/MeCN (19:1) solution and Complex 1a reveals an intense Soret absorption band at m=410 nm (ε=240,000 dm³mol⁻¹cm⁻¹) and a weaker Q band at λₘₐₓ=520 nm. The gold(III) solution was monitored over 48 h at room temperature and less than 10% decrease in the Soret band intensity was observed without any significant spectral shift in both the Soret and Q-bands. The results are shown in FIG. 3.

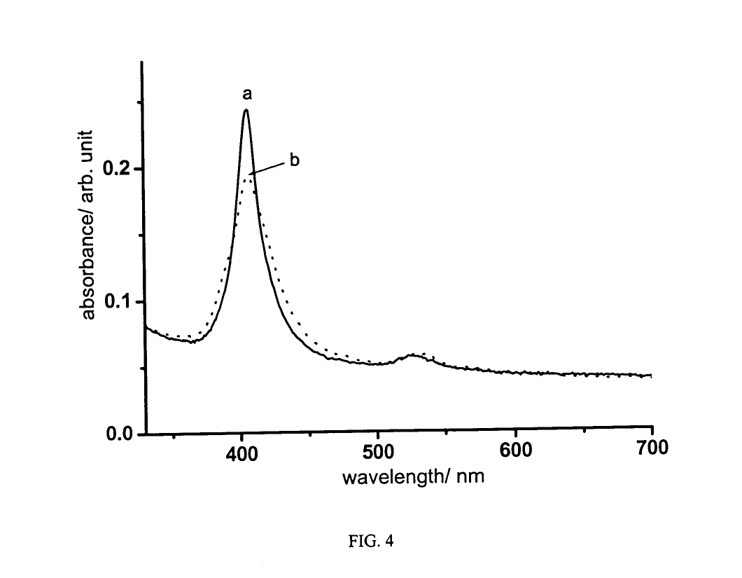

However, in the presence of glutathione (2 mM in Tris buffer/MeCN=19:1), the Soret band intensity of Complex 1a was found to decrease by about 27% with concomitant band broadening upon standing at room temperature over 48 h (FIG. 4). Yet, no significant spectral shift and formation of new absorption bands were observed. Careful examination of the Q band region did not reveal any absorption peaks corresponding to the presence of the free base porphyrin (λₘₐₓ=514, 548, 590 and 640 nm), and thus demetallation can be excluded. Unlike Complex 1a, the UV-vis spectrum of Complex 1j did not show any significant changes over 72 h under an identical buffer medium containing glutathione (2 mM), i.e., no significant changes in the Soret band intensity and band shape were observed.

UV-vis studies also showed that Complexes 1j and 1k were stable in Tris buffer saline (TBS; pH 7.2) for over 48 h at room temperature. Moreover, no significant UV-vis spectral changes were observed when treating 1j/1k with excess GSH. This finding confirms the excellent solution stability of 1j and 1k in physiologically related medium.

Likewise, TBS-MeCN (9:1) (pH 7.2) solutions of the Schiff-base (2a) and bis(pyridyl)carboxamide (3c) complexes exhibited no significant spectral changes after standing at room temperature for 4 h. The results indicates that the gold(III) Schiff-base (2a) and bis(pyridyl)carboxamide complexes are stable. However, addition of GSH to the TBS solutions of Complexes 2a and 3c resulted in spontaneous spectral changes, accompanied by formation of some colloidal gold and light yellow precipitates. The precipitates are soluble in common organic solvent such as THF, CHCl₃ and DMSO but insoluble in aqueous solution. FAB-MS analysis of the precipitates revealed the molecular ions of the free H₂Salen (m/z=267) for Complex 2a and H₂N₄ (m/z=498) for Complex 3c. And yet, ESI-MS analysis of the remaining solutions did not reveal any ion species corresponding to the molecular ions of Complexes 2a (m/z=463) and 3c (m/z=694). The results indicate that Complexes 2a and 3c underwent extensive demetallation and reduction of gold(III) to colloidal gold upon reaction with GSH.

¹H-NMR experiments. NMR experiments were carried out with a d₃-MeCN/D₂O (1:9) solution of Complex 1a (0.5 mM) in the presence of 4 mole equivalent of GSH as described by Sun et al., Eur. J. Biochem. 267:5450-5457 (2000). The pH was adjusted to 7.4 with NaOD, and the solution was degassed for 10 min by bubbling with nitrogen to minimize oxidation of GSH. Similar experimental setup was done for Complex 1j, except this analysis was carried out in D₂O only. The electronic spectra of the resulting mixtures was then monitored for 72 h.

The mixture of Complex 1a/GSH (2 mM) mixture was monitored by ¹H NMR in D₂O/CD₃CN mixture (9:1 v/v), and no significant spectral changes were observed for over 72 h at room temperature. It is known that the chemical shifts of the pyrrolic protons are sensitive to the oxidation states of the gold center, and reduction of Au(III) to Au(I) or Au(0) is expected to be accompanied by spectral changes in the pyrrolic region. However, in this work, we did not observe any significant changes of the spectral pattern in the aromatic region (δ_{H} 7.2-8.4 ppm) for Complexes 1a and 1j, and therefore reduction of Au(III) to Au(I) or Au(0) by GSH is untenable. This finding strongly suggests that the gold(III) porphyrin is stable in physiological conditions, even in the presence of mild reducing agents.

Similarly, no significant ¹H NMR spectral changes were observed when treating 1j/1k with excess GSH. This finding confirms the excellent solution stability of 1j and 1k in physiologically related medium.

Glutathione can be oxidized to form a disulfide bridged dimer (GSSG) with a mild oxidizing agent such as gold(III) complex. A previous study by Sun et al., Eur. J. Biochem. 267:5450-5457 (2000) established that GSSG is characterized by a doublet absorption peak at δ_{H} 3.30 ppm, assignable to β-CH₂ group as depicted in Scheme 5. Scheme 5. Characteristic proton resonance absorptions for (a) GSH and (b) GSSG in D₂O

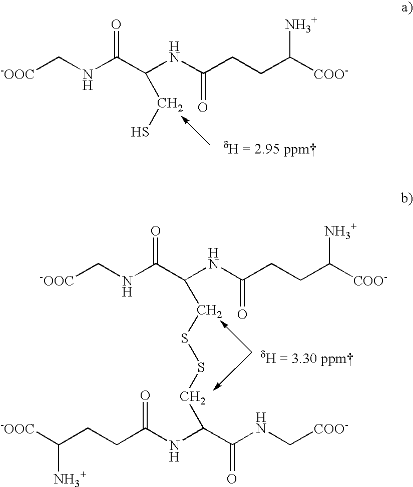

In our ¹H NMR study, we did not observe any doublet proton absorption around the 3.30 ppm region corresponding to the GSSG formation or binding of GSH to the gold(III) center. The results of the NMR studies indicate that illustrative Complexes 1a-1k are stable under physiologically relevant conditions. The result indicates that the Gold(III) Complexes of the invention are stable under physiologically relevant conditions

With regard to the Soret band broadening as noted in Section above, we found that addition of acetone (0.4 ml) to the Tris buffer solution containing Complex 1a led to complete recovery of the Soret band to its initial band shape without any changes in the extinction coefficient (512,000 dm³mol⁻¹cm⁻¹). We reason that the observed band broadening is probably due to aggregation of the gold(III) porphyrin complex in aqueous buffer solution.

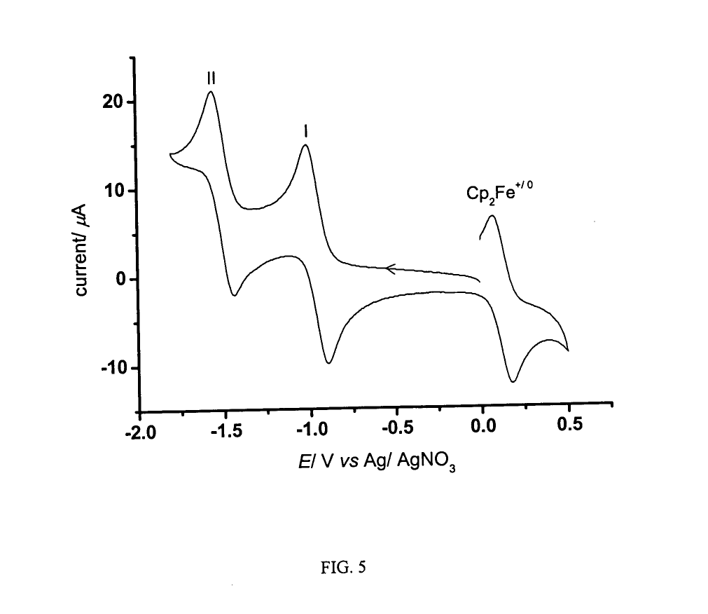

Electrochemical measurements. Electrochemical measurements were performed at room temperature after purging with nitrogen using 0.1 M tetrabutylammonium hexafluorophosphate (TBAP)/acetonitrile as supporting electrolyte. The working electrode was a glassy carbon (Atomergic Chemetal V25, geometric area of 0.35 cm²) electrode and the counter electrode was platinum gauze. A nonaqueous Ag/AgNO₃ (0.1 M in acetonitrile) reference electrode was contained in a separate compartment connected to the test solution via fine sintered glass disks. The ferrocenium/ferrocene couple was used as the internal standard. The redox properties of the gold(III) porphyrin complexes in non-aqueous medium (CH₂Cl₂ with 0.1 M nBu₄NPF₆) have been studied by cyclic voltammetry with glassy carbon and platinum as the working and counter electrodes, respectively. FIG. 5 shows the cyclic voltammogram of Complex 1a, which exhibits two reversible reduction waves at −1.00 and −1.48 V vs Cp₂Fe^{+/0}. Table 3 shows the electrochemical data for Complexes 1e, 1d, 1a, 1b and 1c, i.e., tetrakis(para-Y-substituted phenyl)porphyrins where Y=—Cl, —Br, —H, —Me and —MeO, respectively. Apparently, electron-withdrawing groups such as —Br— and —Cl promote reduction of the III) porphyrin as reflected by a smaller E⁰ values for Complexes 1d and 1e, respectively. Complexes 1b and 1c with electron-donating groups, i.e., -methyl and -oxy, respectively, display larger E⁰ values, indicating that reduction is less favored. Interestingly, both the reduction couples I and II show a parallel substituent effect on their electrochemical potential values. This can be illustrated by plotting the [symbol] E° ( [symbol] E°=E°_{Y}−E°_{H}) values of the couples against each other over a series of para-substituents; a straight line with a slope of 1.00 was obtained (graph not shown). The effect of para-substitution on the electrochemical potentials has been examined by Hammett correlation analysis. Fitting (by least-squares method) the [symbol] E° values with the 4σₚ values, where cup is the Hammett substituent constant, a straight line (R=0.97) was obtained, albeit with some deviation from linearity.

TABLE 3
Reduction Potentials Complexes of Formula (I), containing -phenyl substituents.

| entry | Complex | Couple I ^{a}/V | Couple II ^{a}/V | ΔE₁o ^{b}/V | ΔE2⁰ _{b}/V | σ_{JJ}^{• c} | σₚ ^{d} |
|---|---|---|---|---|---|---|---|
| 1 | 1e | −0.96 | −1.44 | +0.44 | +0.44 | 0.22 | 0.34 |
| 2 | 1d | −0.97 | −1.45 | +0.03 | +0.03 | 0.23 | 0.26 |
| 3 | 1a | −1.00 | −1.48 | 0 | 0 | 0 | 0 |
| 4 | 1b | −1.01 | −1.49 | −0.01 | −0.01 | 0.15 | −0.17 |
| 5 | 1c | −1.02 | −1.51 | −0.02 | −0.02 | 0.23 | −0.27 |

^{a}Couple I and couple II are the first and the second reduction couples of [AuIII(p-Y-TPP)]Cl Complexes, respectively.
^{b}ΔE⁰ = E⁰_{Y} − E⁰_{H} = ρ_{JJ}^{•} Σσ_{JJ}^{•} + ρₚΣσₚ = 0.00649 Σσ_{JJ}^{•}+ 0.0238 Σσₚ (|ρ/ ρ_{JJ}^{•}| = 3.67).
^{c}See ref. Jiang, X.-K. Acc. Chem. Res. 1997, 30, 283-289.
^{d}See ref. Isaacs, N. S. Physical Organic Chemistry, Longman Scientific & Technical. Harlow, Essex, England 1995.

The results indicate that the electronic characteristics of the illustrative Gold(III) Complexes can be “tuned” by appending different groups to the ligand, implying that the electronic characteristics of the Gold(III) Complexes can be tuned to improve their effectiveness as inhibitors of reverse transcriptase of Human Immunodeficiency virus-1 or as anti-tumor agents.

### EXAMPLE 3

### Cytotoxicity Studies Using the Gold(III) Complexes

Example 3 describes the results of cytotoxicity studies using illustrative Gold(III) Complexes 1a-1k, 2a-2c and 3a-3c.

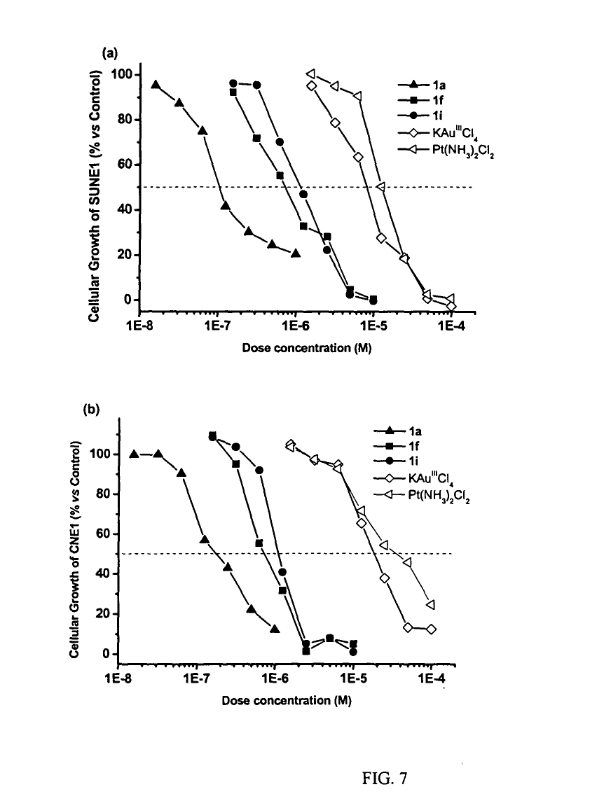

The anti-cancer activities of the gold(III) porphyrin complexes were evaluated toward some established human cancer cell lines: promyelocytic leukemia (HL-60), hepatocellular carcinoma (HepG2), cervix epitheloid carcinoma (HeLa), epidermoid carcinoma (KB-3-1) and its multi-drug resistant variant (KB-V1), nasopharyngeal carcinoma (SUNE1) and its cisplatin-resistant variant (CNE1). The experiments were carried out by following the MTT procedure (see Mosmann et al., J. Immunol. Methods 65:55-63 (1983). The IC₅₀ values of the gold(III) porphyrin complexes together with that of cisplatin, and KAuCl₄ are provided in Table 4a; and the IC₅₀ values of the gold(III) Schiff-base and bis(pyridyl)carboxamide complexes are shown in Table 4b. The cytotoxicity profiles of some selected gold(III) porphyrin complexes toward SUNE1 and its cisplatin-resistant variant CNE1 are also shown in FIG. 7.

TABLE 4a
In Vitro Growth Inhibition of Selected Cancer Cell Lines by Gold (III) Porphyrin
Complexes (a) and Gold (III) Schiff-base and Bis(pyridyl)carboxamide Complexes (b).

| (a) | | IC₅₀ (μM) | | | | | | |
|---|---|---|---|---|---|---|---|---|
| entry | Complex | KB-3-1 ^{a} | HL-60 | HepG2 | SUNE1 | HeLa | KB-V-1 | CNE1 |
| 1 | 1a | 0.20 | 0.73 | 0.34 | 0.11 | 0.28 | 0.12 | 0.17 |
| 2 | 1b | 0.41 | 1.53 | 1.11 | 1.09 | 0.52 | 1.15 | 0.99 |
| 3 | 1c | n.d.^{b} | 0.88 | 0.76 | 0.68 | 0.66 | n.d.^{b} | 0.77 |
| 4 | 1d | 1.29 | 1.12 | 1.21 | 0.74 | 0.89 | 0.64 | 0.98 |
| 5 | 1e | 0.50 | 0.87 | 0.92 | 0.34 | 0.69 | 0.23 | 0.41 |
| 6 | 1f | 0.87 | 0.74 | 1.83 | 0.68 | 0.68 | 0.71 | 0.73 |
| 7 | 1g | n.d.^{b} | 1.45 | 2.31 | 2.11 | 3.18 | n.d.^{b} | 1.45 |
| 8 | 1b | 0.99 | 0.75 | 1.17 | 1.18 | 0.96 | 1.14 | 2.06 |
| 9 | 1i | 0.91 | 1.24 | 1.09 | 0.96 | 1.06 | 0.99 | 1.37 |
| 10 | 1j | >50 | >50 | >50 | >50 | >50 | >50 | >50 |
| 11 | 1k | >50 | >50 | >50 | >50 | >50 | >50 | >50 |
| 12 | nBu₄N[AuCl₄] | 10.3 | 9.71 | 11.2 | 9.94 | 8.41 | n.d.^{b} | 11.5 |
| 13 | Pt(NH₃)₂Cl₂ | 13.2 | 16.8 | 13.5 | 12.6 | 11.2 | 10.2 | 40.8 |
| 14 | [Zn(TPP)] | >50 | >50 | >50 | >50 | >50 | n.d.^{b} | >50 |

| (b) | | IC₅₀ (μM) |
|---|---|---|
| entry | Complex | HeLa |
| 1 | 2a | 11.8 |
| 2 | 2b | 9.71 |
| 3 | 2c | 16.5 |
| 4 | 3a | 33.9 |
| 5 | 3b | 26.2 |
| 6 | 3c | 10.8 |
| 7 | Pt(NH₃)₂Cl₂ | 11.2 |

^{a} Human Oral Epidermoid Carcinoma (KB-3-1); Human Promyelocytic Leukemia (HL-60); Human Hepatocellular Carcinoma (HepG2); Human Nasopharyngeal Carcinoma (SUNE1); Human Cervix Epitheloid Carcinoma (HeLa); Human Oral Epidermoid Carcinoma (multi-drug resistance, KB-V-1); Human Nasopharyngeal Carcinoma (cisplatin resistance, CNE1).

All the gold(III) porphyrin complexes studied in this work, except complexes 1j and 1k, show relevant cytotoxic activities with IC₅₀ values falling into the micromolar range (0.1-15 μM). Among them, Complex 1a was found to be the most cytotoxic with the smallest IC₅₀ values of 0.1-0.8 μM.

The cytotoxic properties of the [Au^{III}(para-Y-TPP)]C1 Complexes 1e, 1b, 1a, 1d and 1e (Y=MeO, Me, H, Br and Cl, respectively) were examined, and their cytotoxicities are largely unaffected by electronic substituent effect.

Compared to the other gold(III) porphyrins, Complexes 1j and 1k are non-cytotoxic as manifested by their rather large IC₅₀ values >50 μM. This may be ascribed to the highly polar and hydrophilic groups that rendered the compounds unable to pass through the hydrophobic cell membrane.

In this study, all the gold(III) porphyrin complexes excepted higher cytoxicities toward the studied cell lines than cisplatin. This is shown in Table 5, where the ratio of Cisplatin IC₅₀/Gold(III) Porphyrin Complex IC₅₀ for a given cell line.

TABLE 5
IC₅₀ Ratio [Cisplatin/Gold (III) Porphyrin Complexes]

| | | IC₅₀ ratio [cisplatin/ | | | | |
|---|---|---|---|---|---|---|
| | | gold (III) porphyrin complexes] | | | | |
| entry | Compound | KB-3-1 | HeLa | SUNE1 | KB-V1 | CNE1 |
| 1 | 1a | 17.8 | 40.0 | 114.5 | 25.1 | 240.2 |
| 2 | 1b | 32.1 | 21.5 | 11.5 | 18.4 | 41.2 |
| 3 | 1c | n.d.^{a} | 17.0 | 18.5 | n.d.^{a} | 53.0 |
| 4 | 1d | 10.23 | 12.6 | 17.0 | 8.96 | 41.6 |
| 5 | 1e | 26.4 | 16.2 | 37.1 | 20.4 | 99.5 |
| 6 | 1f | 15.1 | 16.5 | 18.5 | 5.62 | 55.9 |
| 7 | 1g | n.d.^{a} | 3.52 | 5.97 | n.d.^{a} | 28.1 |
| 8 | 1h | 13.3 | 11.7 | 10.7 | 6.12 | 19.8 |
| 9 | 1i | 14.5 | 10.56 | 13.1 | 7.31 | 29.8 |

The results in Table 5 also show that except for Complexes 1i, 1j and 1k, the gold(III) porphyrin complexes show at least ten times higher cytotoxicity than cisplatin. Notably, Complex 1a exhibits significant cytotoxicity (IC₅₀=0.17 μM) against the cisplatin-resistant nasopharyngeal carcinoma (CNE1) cell lines; the ratio of the IC₅₀ values for Complex 1a and cisplatin was determined to be 240 (see Table 5). The important role of the gold(III) ion on the cytotoxic properties of the metalloporphyrin is reflected by the fact that the [Zn^{II}TPP] complex is at least 100-fold less cytotoxic (IC₅₀>50 μM) than Complex 1a against an identical panel of cell lines. By comparing the cytotoxicity profile of Complex 1a with that of KAu^{III}Cl₄, the porphyrin ligand appears to have a significant role in promoting the cytotoxicity of gold(III) complex. The results indicate that KAu^{III}Cl₄ is about 40 times less potent than Complex 1 a in killing oral epidermoid and nasopharyngeal cancer cells. As noted earlier, the Au(3+) ion is unstable under physiological conditions and undergoes reduction to colloidal gold. Without being bound by theory, we believe that the porphyrin ligand stabilizes the Au(3+) center and serves as a carrier to bring the toxic metal to its cellular target.

The cytotoxicities of the other gold(III)porphyrin complexes toward the cisplatin-resistant cell line (CNE1) and the cisplatin-sensitive cell lines (SUNE1) the cisplatin-resistant cell line (CNE1) and the cisplatin-sensitive cell lines (SUNE1) are similar to that of Complex 1ar; the ratio of IC₅₀ (CNE1/SUNE1) values are close to unity. The results suggests that the mechanism for the cytotoxicity of the gold(III) porphyrin complexes is different from that of cisplatin.

### EXAMPLE 4

### Interaction of Gold(III) Porphyrin Complexes with DNA

Example 4 describes the results of studies showing the interaction of illustrative Gold(III) Complexes 1a, 1g, 1h and 1j with DNA.

Restriction Endonuclease Fragmentation Assay: Restriction endonuclease fragmentation assay was used to detect potential interaction of Complex 1a with DNA. Binding of the metalloporphyrin onto a duplex DNA such as plasmid DNA (pDR2) is expected to occupy the binding site of the restriction enzyme ApaI, thereby inhibiting the enzymatic DNA digestion. See Ikeda et al., J. Am. Chem. Soc. 104:296-297 (1982).

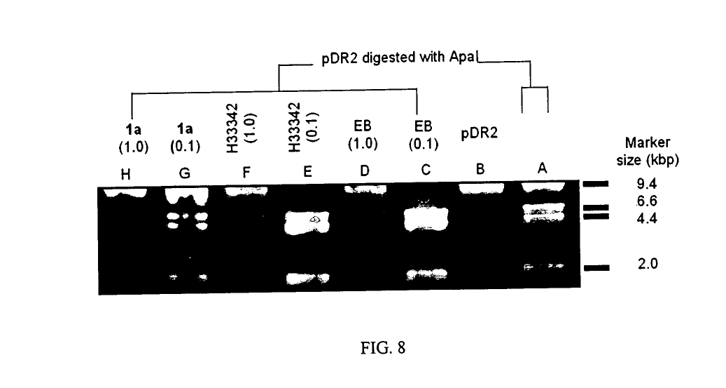

FIG. 8 shows the results of electrophoresis of pDR2 after restriction enzyme digestion in the absence and presence of Complex 1a. The undigested DNA (lane A) shows a fragment at around 9.6 kbp corresponding to the circular DNA. After ApaI digestion, (lane B), three bands corresponding to 6.6, 4.4 and 2.0 kbp were observed. In the presence of Complex 1a at 1:1 (Complex 1a :DNA bp) ratio, inhibition of the DNA digestion was observed (lane D). Yet, partial DNA digestion was observed at a lower molar concentration of Complex 1a (lane C) (Complex 1a:DNA=0.1:1). For comparison, the presence of ethidium bromide (a classical intercalator; lane F) and Hoechst 33342 (a minor groove binder; lane H) led to complete inhibition of the DNA digestion. The results suggest that Complex 1a would bind to DNA and inhibit enzymatic digestion by restriction endonucleases.

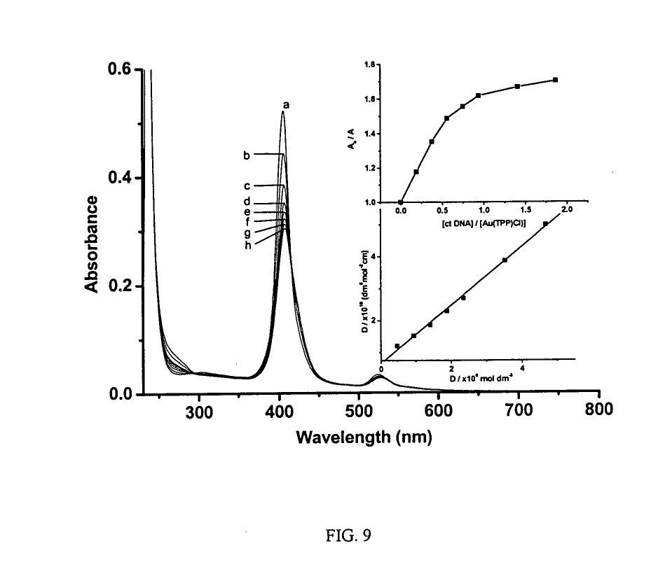

Absorption Titration. The binding interaction of the gold(III) porphyrins to duplex DNA has been studied by UV-vis spectroscopy. As shown in FIG. 9, addition of calf thymus DNA (0-5 μM) to Complex 1a in a Tris buffer solution containing 2% DMSO induced a significant hypochromicity (44%) of the Soret band, accompanied with a small bathochromic shift of 5 nm. For the Q-band at 520 nm, 40% hypochromicity and 6 nm bathochromic shift were also observed. It is noteworthy that the spectral changes displayed clear isosbestic points at 290, and 425 nm, which remain unchanged throughout the spectroscopic titration experiment.

Inset A of FIG. 9 shows the plot of Iₒ/I versus [ctDNA]/[1a], where Aₒ and A are the Soret band absorbances of Complex 1a in the absence and presence of ctDNA, respectively. As shown in Inset A, the Aₒ/A value increases linearly with the rising [ctDNA]/[Complex 1a]ratio from 0 to 1.0; no further hypochromicity of the Soret band was observed when the [ctDNA]/[Complex 1a]is larger than 1.0. This result indicates that the binding stoichiometry (Complex 1a:number of base pairs) is 1:1.

The intrinsic binding constant (K_{b}) for Complex 1a toward calf thymus DNA was determined according to equation 1:

$$\frac{[ ctDNA ]}{Δ {ϵ}_{ap}} = \frac{[ ctDNA ]}{Δ ϵ} + \frac{1}{( Δ ϵ \times {K}_{b} )} \qquad ( 1 )$$

where [ctDNA] is the concentration of DNA, Δεₐₚ═|ε_{A}−ε_{B}| in which ε_{A}=A_{obs}/[complex], and Δε=|ε_{B}−ε_{F}|. ε_{B} and ε_{F} correspond to the extinction coefficient of DNA-bound gold(III) porphyrin and the free gold(III) porphyrins, respectively. Using the absorbance data of the Soret band, the plot of [ctDNA]/Δεₐₚ vs [ctDNA] gives a straight line (R=0.99) with a slope of 9.15×10⁻⁶, which corresponds to the reciprocal of the Δε value. From the intercept of the plot, the intrinsic binding constant for Complex 1a to calf thymus DNA was determined to be (2.79±0.34)×10⁶ dm³ mol⁻¹.

Similarly, Complexes 1g, 1h and 1j also produced comparable isosbestic spectral changes upon interaction with calf thymus DNA (data not shown). Significant hypochromicity (20-50%) and bathochromic shift (3-6 nm) of the Soret bands were observed. On the basis of the Aₒ/A vs [ctDNA]/[metal complex] plot, the stoichiometric binding ratio for the gold(III) complexes with the number of base pairs were also found to be 1:1. Again using the equation 1, their intrinsic binding constants to calf thymus DNA were determined, and the values are tabulated in Table 6.

TABLE 6
DNA Binding Parameters for Selected Classes of Gold(III)
Porphyrin Complexes in Tris Buffer at Various Temperatures

| | | intrinsic binding | |
|---|---|---|---|
| | | constant (K_{b}), | |
| Complex | temperature, K | dm³mol⁻¹ | hypochromicity, % |
| 1a | 292.8 | 2.79 × 10⁶ | 53.4 |
| | 298.0 | 2.14 × 10⁶ | 50.3 |
| | 302.3 | 1.98 × 10⁶ | 49.5 |
| | 307.2 | 1.55 × 10⁶ | 47.1 |
| | 311.6 | 1.35 × 10⁶ | 46.3 |
| | 316.5 | 0.94 × 10⁶ | 43.4 |
| | 321.9 | 0.82 × 10⁶ | 42.5 |
| 1g | 291.8 | 0.73 × 10⁶ | 18.2 |
| | 297.6 | 0.58 × 10⁶ | 18.0 |
| | 301.4 | 0.47 × 10⁶ | 16.5 |
| | 306.1 | 0.42 × 10⁶ | 10.1 |
| | 311.7 | 0.34 × 10⁶ | 8.7 |
| 1h | 291.4 | 4.14 × 10⁶ | 30.1 |
| | 296.8 | 2.73 × 10⁶ | 29.2 |
| | 302.2 | 1.51 × 10⁶ | 28.0 |
| | 305.5 | 1.27 × 10⁶ | 26.1 |
| | 311.2 | 0.96 × 10⁶ | 24.4 |
| 1j | 291.3 | 0.49 × 10⁶ | 47.8 |
| | 297.2 | 0.36 × 10⁶ | 45.9 |
| | 303.7 | 0.24 × 10⁶ | 43.1 |
| | 307.2 | 0.21 × 10⁶ | 41.1 |
| | 311.9 | 0.19 × 10⁶ | 40.6 |

The intrinsic binding constants obtained in this work are comparable to reported values (10⁴ to 10⁶ dm³mol⁻¹) of some related metalloporphyrin complexes disclosed by Lugo-Ponce et al., Coord. Chem. Rev. 208:169-191 (2000); Tjahjono et al.; J. Inorg. Biochem. 85:219-228 (2001); and Han et al., J. Inorg. Biochem. 91:230-236 (2002).

In a control experiment using sodium dodecyl sulfate (0-5 μM) instead of DNA, similar UV-vis spectral changes for Complexes 1a and 1g were observed (48% hypochromicity and 6 nm bathochromic shift) (data not shown). This result suggests that the observed spectral changes with the calf thymus DNA is probably due to electrostatic binding of the cationic metal complexes with the polyanionic DNA phosphate backbone. See Neidle, S. DNA structure and recognition; Oxford University Press: Oxford, 1994; 74-79. However, when Complexes 1h and 1j was titrated with sodium dodecyl sulfate, minor hypochromicity (<10%) of their Soret bands was observed, suggesting that other binding modes for the gold complexes may be more important than electrostatic binding with the DNA backbone.

The effect of temperature on the intrinsic binding constants was investigated. Using the van't Hoff equation (2):

In K=−(ΔH/RT)+(ΔS/R) (2)

the linear plot of In K vs 1/T yields the thermodynamic parameters for the DNA binding interactions. As shown in Table 7, the DNA binding interactions with Complexes 1a, 1 g, 1 h and 1j show a comparable negative ΔH value (−7 to −13 kcalmol⁻¹), indicative of favorable enthalpic changes.

TABLE 7
Thermodynamic Parameters for the Binding of Complexes
1a, 1g, 1h and 1j to Calf Thymus DNA

| entry | Complex | ΔG° (298 K) | ΔH° | ΔS° |
|---|---|---|---|---|
| 1 | 1a | −8.7 ± 0.4 | −8.0 ± 0.5 | +2.4 ± 0.3 |
| 2 | 1g | −7.8 ± 0.5 | −7.0 ± 0.4 | +2.7 ± 0.7 |
| 3 | 1h | −8.2 ± 0.7 | −13.8 ± 1.2 | −17.3 ± 0.7 |
| 4 | 1j | −7.5 ± 0.4 | −8.5 ± 0.6 | −3.4 ± 0.5 |

However, Table 7 shows that there are notable variations for the ΔS values among the gold(III) complexes. For Complexes 1a and 1g, the DNA binding reactions are associated with small positive entropic changes of +2.4 and +2.7 calmol⁻¹K⁻¹, respectively, consistent with increasing disorder upon binding of the complexes to DNA. On the other hand, the binding of DNA to Complexes 1h and 1j produces negative entropic changes (−17.3 cal mol⁻¹K⁻¹ (Complex 1 h), −3.4 calmol⁻¹K⁻¹ (Complex 1j)), indicating a loss of degree of freedom upon binding to DNA. This finding suggests that different binding modes would be expected for these two groups of gold(III) complexes.

Viscosity evaluation. The binding interaction of the gold(III) porphyrins can be further investigated by examining the change of DNA viscosity upon binding of the metalloporphyrin. If the metalloporphyrin binds to DNA by intercalation, this would cause chain elongation, unwinding and stiffening of the DNA helix as a consequence of untwisting of the base pairs and helical backbone needed to accommodate the intercalator.

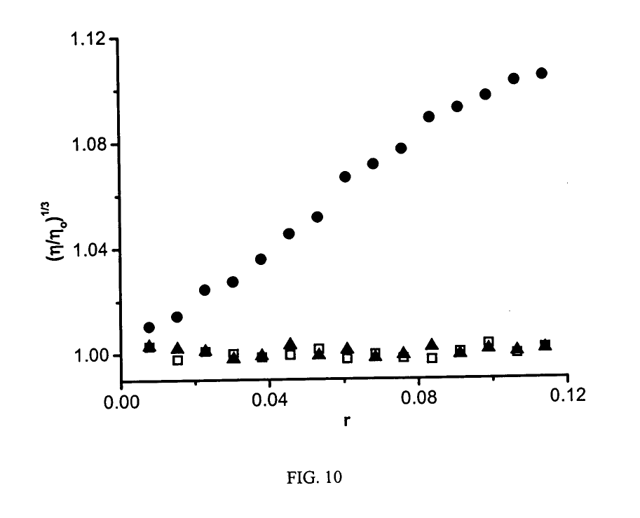

Based on the theory of Cohen and Eisenberg (Cohen et al, Biopolymers 8:45-55 (1969)), the viscosity data were plotted as (η/ηₒ)^{1/3} vs the binding ratio, r=[DNA]/[Complex 1a]. The plot is shown in FIG. 10. For comparison, the viscosity profiles for ethidium bromide (intercalator) and Hoechst 33342 (minor groove binder) are also shown is FIG. 10. As anticipated, the intercalator, i.e., ethidium bromide, increases the viscosity of the DNA by increasing its hydrodynamic length. Yet, both Complex 1a and Hoechst 33342 do not lengthen the DNA and exhibit no appreciable change in viscosity of the DNA. The result suggests that Complex 1a is unlikely to interact with DNA through intercalation.

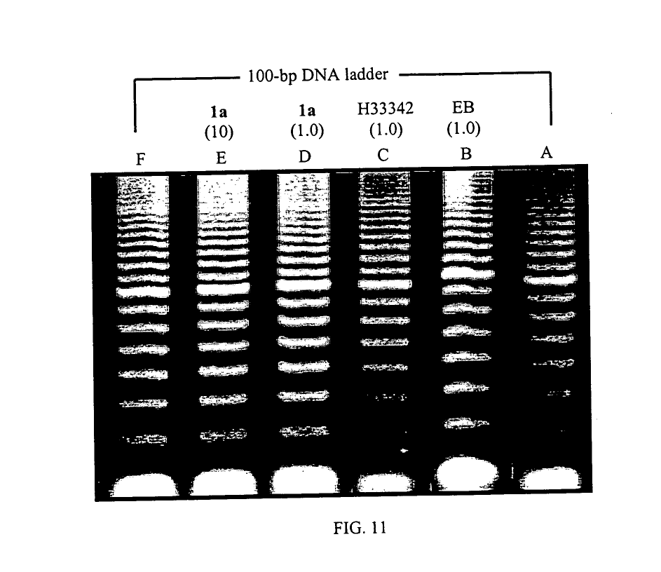

Gel mobility shift assay. Additional evidence for non-intercalative binding of Complex 1a to DNA was obtained by gel mobility shift assay. Elongated DNA will have lower mobility compared to the free DNA in the agarose gel electrophoresis. FIG. 11 shows the results of the gel mobility shift assay. The 100-bp PCR, alone or treated with Complex 1a, Hoechst 33342 (H33342) and ethidium bromide, were mixed and resolved by agarose gel electrophoresis. It is apparent that those DNA samples treated with H33342 (lane C) and Complex 1a (lane D and E) showed similar mobility shift to the untreated 100-bp PCR. In contrast, lane B showed the tailing effect, which was due to the intercalation of the ethidium bromide to the DNA base pairs. The result suggests that Complex 1a is unlikely to bind to DNA by intercalation.

### EXAMPLE 5

### Apoptosis Studies

Example 5 describes the results of a cell morphology study using illustrative Gold(III) Complex 1a.

Confocal Microscopic Experiment. By examining cell morphology using scanning confocal microscopy, the type of cell death can be recognized. The early phase of apoptosis is characterized by the formation of apoptotic bodies while the integrity of the cell membrane is maintained without cell lysis. On the contrary, necrosis is characterized by cell lysis with the internal cellular components such as DNA being exposed. Using suitable staining agents (ethidium bromide (EB) for DNA and acridine orange (AO) for cell membrane) normal, necrotic and apoptotic cells can be distinguished by laser confocal microscopy. See Chan et al., Inorg. Chem. 41:3161-3171 (2002).

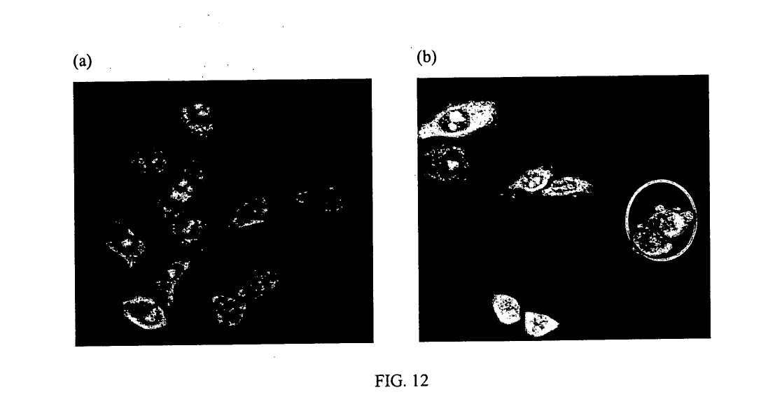

The picture of the cells is depicted in FIG. 12. After incubation of the HeLa cells with Complex 1a for 15 h, living cells and apoptotic cells were observed, and several apoptotic bodies can be seen (marked by circles) in several cells. Importantly, no cellular DNA was exposed due to necrosis.

Flow cytometry: The gold(III) porphyrin-induced apoptosis of the HeLa cell line has been further examined by flow cytometry. During the early phase of apoptosis, cells lose the asymmetry of their membrane phospholipids, although the integrity of the cell membrane is maintained. A negatively charged phospholipid, named phosphatidylserine (PS), which is located in the inner part of the plasma membrane, becomes exposed to the cell surface. A phospholipid-binding protein, annexin V (green emissive upon binding), with high affinity binds preferentially to PS. Apoptotic cells therefore can be stained by annexin V before the dying cell changes its morphology and hydrolyzes its DNA. On the other hand, both DNA and the PS would be exposed for necrotic cells. In this case, propidium iodide (PI, red emissive upon binding), can be used to stain selectively the naked DNA. Therefore, both apoptotic and necrotic cells can be discriminated after adding these two staining reagents. For apoptotic cells, only annexin V would stain; for necrotic cells, both annexin V and PI would stain.

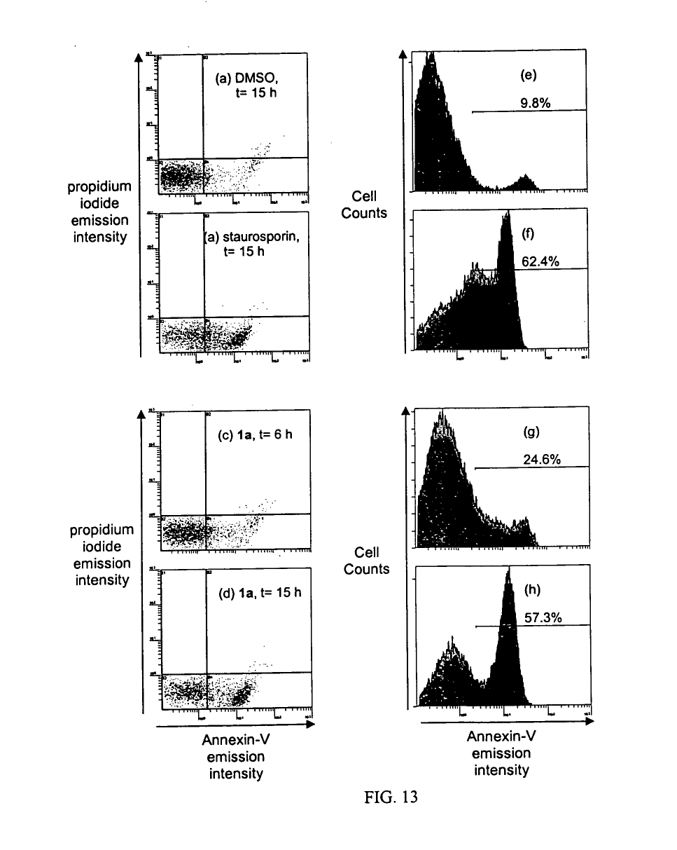

In this experiment, annexin V and PI were added to cell cultures that had either already exposed to Complex 1a (6 and 15 h) or straurosporine (15 h). For comparison, a control cell culture experiment was performed in the absence of Complex 1a and straurosporine. The results (FIG. 13a) show that in the absence of any drug compounds, more than 90% of the cells were neither stained by annexin V α-axis emission) nor PI (y-axis emission), indicating that over 90% of the population were living cells. Upon treatment with Complex 1a or straurosporine, the magnitude of the annexin V emission was enhanced, indicating the onset of apoptosis. The progress of apoptosis induced by Complex 1a was observed by monitoring the magnitude of the annexin V emission. The results in FIG. 13 show that 13, 24% of the cells underwent apoptosis after 6 h (13c,g) of incubation; 57% of the cells died via apoptosis after 15 h (13d,h) of incubation. Throughout the flow cytometric experiment, <1% of necrotic cells were observed.

Irrespective of the mechanism for induction of apoptosis, the results of the confocal microscopy and flow cytometry study show that Complex 1a, an illustrative Gold(III) Complex of the invention, can induce apoptosis in HeLa cell. Without being limited by theory, it is believed that the treatment of the cancer cells with Complex 1a “nicks” cellular DNA and causes oxidative stress. The results indicated that the Gold(III) Complexes are useful for induction of apoptosis in cancer cells.

### EXAMPLE 6

### Inhibition of HIV-1 Reverse Transcriptase by Gold(III) Porphyrins and Derivatives

Example 6 describes a study showing that illustrative Gold(III) Complexes 1, 1j, 1k, 2a and 3c are useful for inhibiting HIV-1 RT activity.

HIV-1 reverse transcriptase activities were measured by an ELISA method (see Eberle et al, J. Virol. Methods, 40:347-356 (1992) performed by incubating the enzyme with digoxigenin-labeled deoxyuridin-5′-triphosphate (dUTP) and the biotin-labeled analogue during reverse transcription starting from the template/primer hybrid poly(A)•oligo(dT)₁₅. After reverse transcription, the newly synthesized DNA was immobilized onto streptavidin-coated ELISA wells, and the incorporation of the dioxigenin-labeled dUTP was assayed by chemiluminescence method. For example, treatment of HIV-1 RT in Lysis buffer (2 ng, 128.7 μL) with Complex 1j (1.3 μL of 5 mM stock solution) dissolved in TBS at 37° C. produced significant RT activity inhibition (˜95%) after 30 min incubation. For comparison, the drug-free control exhibited 0% inhibition. The results of the study are shown in Table 8.

TABLE 8
Inhibitory Concentration (IC₅₀) of Illustrative Gold(III) Complexes
Against HIV-1 RT and Human Lung Fibroblast Cell Line

| | | IC₅₀/μM (±SE)^{a} | |
|---|---|---|---|
| entry | Complex | HIV-1 RT | CCD-19Lu |
| 1 | 1a | 28.4 ± 1.8 | 0.91 ± 0.21 |
| 2 | 1j | 0.31 ± 0.05 | >100 |
| 3 | 1k | 0.57 ± 0.09 | >100 |
| 4 | 2a | 0.33 ± 0.03 | 38.6 ± 4.43 |
| 5 | 3c | 0.47 ± 0.09 | 43.9 ± 3.25 |
| 6 | AZT | 0.069 ± 0.008 | n.d. |

^{a}Expressed as mean ± SE of at least three determinations.
^{b}.n.d. = not determined.
HIV-1 RT = HIV-1 reverse transcriptase;
CCD-19Lu = human lung fibroblast cells.

The HIV-1 RT inhibitory activity of Complex 1j was found to be dose-dependent, % inhibition (concentration): 95% (50 μM), 76% (10 μM), 67% (5 μM), 58% (1 μM), 53% (0.5 μM) and 38% (0.1 μM). The IC₅₀ value was evaluated to be 0.31±0.05 μM (Table entry 1). The anti-HIV RT activity of Complex 1k was also examined by the ELISA method, and an IC₅₀ value (0.57±0.09 μM) was obtained. For comparison, it is noted that the free-base porphyrins (i.e., [H₂TMPyP]⁴⁺ and [H₂TPPS]⁴⁻) exhibited substantially lower HIV-1 RT inhibition activities than did the corresponding metal complex analogs, Complexes 1j and 1k, respectively. Likewise, the results in Table 8 show that Complexes 1a, 2a and 3c are also effective inhibitors of HIV-1 RT, with their measured IC₅₀ values being 28.4±1.8 μM (1a), 0.33±0.03 μM (2a) and 0.47±0.09 μM (3c).

The Zn complex [Zn^{II}(PP)] (H₂PP=protoporphyrin) is reported to inhibit HIV-1 RT activity in vitro (see Argyris et al., J. Biol. Chem. 274:1549 (1991)), with a measured IC₅₀ value (ELISA assay) of 4.98±0.93 μM. Based on the IC₅₀ values shown in Table 8, [Zn^{II}(PP)] is at least 10-fold less potent for HIV-1 RT inhibition than the Gold(III) Complexes 1j, 1k, 2a and 3c of the present invention.

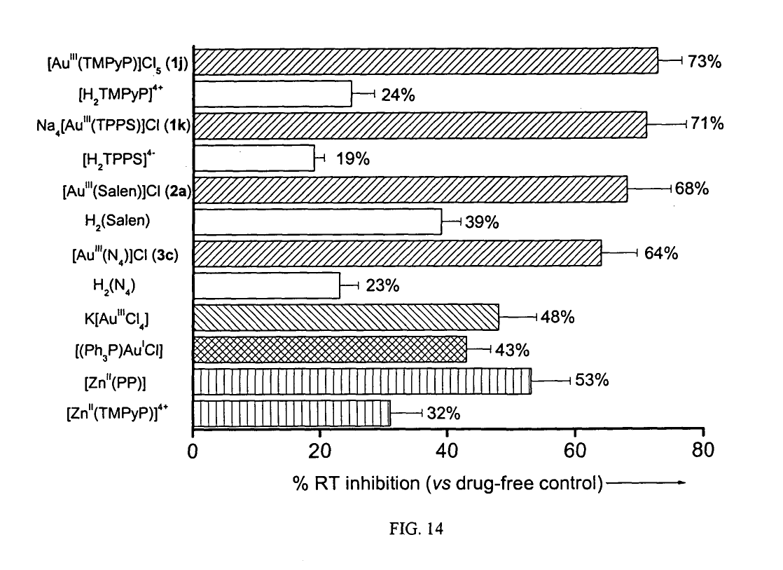

FIG. 14 depicts the results of a comparative study for a series of gold complexes. By fixing the drug concentration at 6 μM, the HIV-1 RT inhibitory activities of the compounds were evaluated by the ELISA method. Up to about 70% HIV-1 RT inhibition was observed for Complexes 1j-k and 2a, whereas Complex 3c was found to affect 64% RT inhibition under identical conditions. The data in FIG. 14 show that the presence of the gold atom is critical for the observed anti-HIV-1 RT activities, since all the free-base ligands are largely inactive (<30%) for RT inhibition. The results in FIG. 14 also show that KAu^{III}Cl₄ was found to result in 48% inhibition of the HIV-1 RT activities, compared to 70% inhibition attained by Complexes 1j, 1k, 2a and 3c. The result indicates that the chelating σ-donor ligands strongly influences the anti-HIV RT activities of the Gold(III) Complexes. Moreover, compared to gold(I) complex [(Ph₃P)Au^{I}Cl], Complexes 1j, 1k, 2a and 3c are more powerful HIV-RT inhibitors in vitro, since treating the enzyme with [(Ph₃P)Au^{I}Cl] (at 6 μM level) produced only 43% inhibition. As noted earlier, [Zn^{II}(PP)] is a less effective HIV-1 RT inhibitor (compare 53% RT inhibition for [Zn^{II}(PP)] to 70% RT inhibition for Complexes 1j, 1k, 2a and 3c under the same conditions). Likewise, the activity of the Zn complex [Zn^{II}(TMPyP)]Cl₄ was found to produce only 32% RT inhibition compared to, e.g., about 70% for Complex 1j at 6 μM level. The results indicate that the gold(III) atom plays a critical role in the RT inhibition activity.

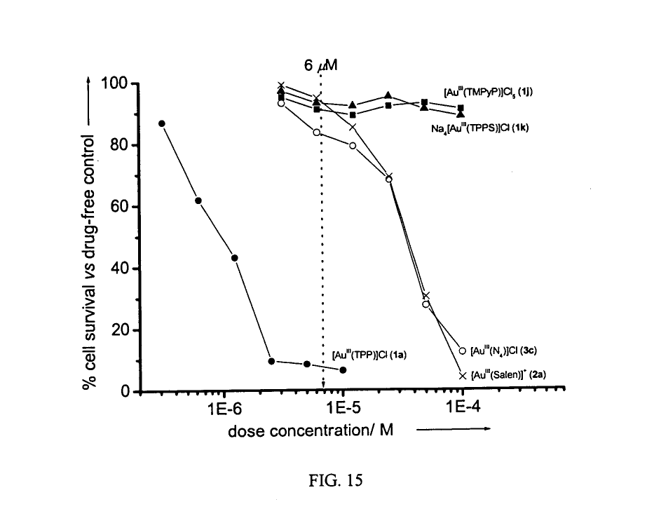

Acute cytotoxicities of the gold(III) complexes to normal cells were evaluated using human lung fibroblast cells (CCD-19Lu) as model cell line. The percentages of cell survival at various doses of the gold complexes were determined by the MTT assay, and the toxicities profiles are shown in FIG. 15. The results suggest that Complexes 1j and 1k do not exhibit significant acute cytotoxicities to the fibroblast cells, with >90% cell survival being registered at drug concentration up to 96 μM, which is 16-fold of their IC₇₀ values (about 6 μM) for HIV-1 RT inhibition. However, 10% cell survival was observed when treating the fibroblast cells with Complexes 2a and 3c at 96 μM level. At 2 μM, Complex 1a produced severe cytotoxicity, with 90% cell death being observed (c.f. IC₅₀=28 μM for HIV-1 RT inhibition).

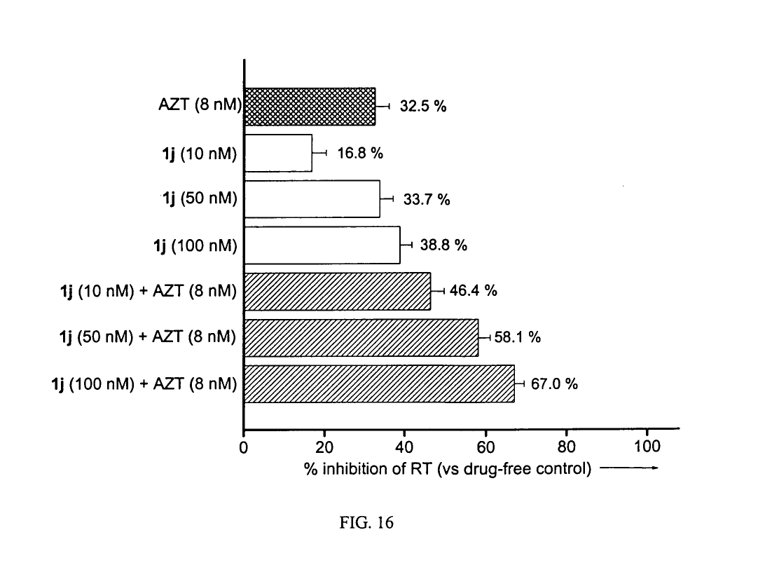

Complex 1j was chosen for further examination based on its solution stability and acute cytotoxicity profile. The HIV-1 RT inhibitory activities of Complex lj alone and in combination with AZT are shown in FIG. 16. The results show that only 17-39% enzyme inhibition was achieved when treating HIV-1 RT with Complex 1j (10, 50 and 100 nM) or AZT (8 nM) alone. However, when Complex 1j was used in combination with AZT, a significant additive effect on HIV-1 RT inhibition was observed. Up to 68% enzyme inhibition was attained by employing the “Complex 1j (100 nM)+AZT (8 nM)” combination. The IC₇₀ results show that the combination of Complex 1j with AZT can lower the dosage requirement of the gold(III) porphyrin by 60-fold compared to using Complex 1j alone. Without being limited by theory, it is believed that the synergistic HIV-1 RT inhibition by Complex 1j and AZT results from the gold(III) porphyrin binding to certain sites nearby the active site of the enzyme. For example, [Zn^{II}(PP)]-mediated HIV-1 RT inhibition is reported to be related to binding of the metalloporphyrin to the connection domain sequence 398407 (WETWWTEYWQ) (see Argyris et al., J. Biol. Chem. 274:1549 (1991)). In this work, the UV-vis absorption titration study revealed that Complex 1j binds strongly to the WETWWTEYWQ sequence in aqueous buffer medium. Addition of the peptide (0-5 μM) to Complex 1j produced isosbestic spectral changes (isosbestic points at 314, 417, 508 and 532 nm) with significant hypochromicity (58%) and small bathochromic shift (7 nm) of the Soret band. Using the absorbance data of the Soret band, the linear plot (R=0.99) of [peptide]/Δεₐₚ vs [peptide] gave the intrinsic binding constant (Kb)=(7.9±0.7)×10⁴ dm³mol⁻¹ at 292 K. A comparable Kb value [(1.3±0.1)×10⁵ dm³ mol⁻¹ at 292 K] was obtained for the [Zn^{II}(PP)]-WETWWTEYWQ interaction based on UV-vis absorption titration study. No significant binding of Complex 1k with the peptide was observed based on UV-vis spectral titration. Given that Complex 1k is also an effective HIV-1 RT inhibitor, the poor binding affinity to WETWWTEYWQ may suggest an alternative binding site for the complex.

The present invention is not to be limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited, the entire disclosures of which are incorporated herein by reference.

What is claimed is:

1. A method for induction of apoptosis of cancer cells comprising administering to a patient in need thereof a composition comprising an effective amount of a gold(III) complex of formula: 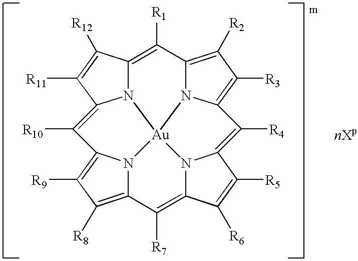
or a pharmaceutically acceptable salt thereof, wherein:
R₁, R₄, R₇ and R₁₀ are each independently —H, -halo, —(C₁-C₆)alkyl or —O(C₁-C₆)alkyl, -(6-membered)aryl or -(5 to 10-membered)heteroaryl, each of which may be substituted with one or more -halo, —(C₁-C₆)alkyl, —O(C₁-C₆)alkyl, —OSO₂ or —NO₂;
R₂, R₃, R₅, R₆, R₈, R₉, R₁₁ and R₁₂ are each independently —H, —(C₁-C₆)alkyl which may be substituted with one or more —C(O)OR₁₃, -halo or ═O groups;
R₁₃ is —(C₁-C₆)alkyl;
each X^{p} is independently a pharmaceutically acceptable counter-ion;
m is an integer ranging from −3 to 5;
p is an integer ranging from −3 to 3;
n is equal to the absolute value of m/p; and
a pharmaceutically acceptable carrier.

2. The method of claim 1, wherein R₂, R₃, R₅, R₆, R₈, R₉, R₁₁ and R₁₂ are each —H.; X^{p} is Cl⁻; m is 1; and n is 1.

3. The method of claim 2, wherein R₁, R₄, R₇ and R₁₀ are each -phenyl.

4. The method of claim 2, wherein R₁, R₄, R₇ and R₁₀ are each -4-methylphenyl.

5. The method of claim 2, wherein R₁, R₄, R₇ and R₁₀ are each -4-methoxyphenyl.

6. The method of claim 2, wherein R₁, R₄, R₇ and R₁₀ are each -4-bromophenyl.

7. The method of claim 2, wherein R₁, R₄, R₇ and R₁₀ are each -4-chlorophenyl.

8. The method of claim 2, wherein R₁, R₄, R₇ and R₁₀ are each -3,4,5-trimethoxyphenyl.

9. The method of claim 2, wherein R₁, R₄, R₇ and R₁₀ are each -3,4,5-trifluorophenyl.

10. The method of claim 1, wherein R₁, R₄, R₇ and R₁₀ are each —H; R₂, R₃, R₅, R₆, R₈, R₉, R₁₁ and R₁₂ are each -ethyl; X^{p} is Cl⁻; m is 1; and n is 1.

11. The method of claim 1, wherein R₁, R₄, R₇ and R₁₀ are each —H; and R₂ and R₁₁ are each -ethyl; R₃, R₅, R₉ and R₁₂ are each -methyl; R₆ and R₈ are each -methyl-3-propanoate; X^{p} is Cl⁻; m is 1; and n is 1.

12. The method of claim 1, wherein R₁, R₄, R₇ and R₁₀ are each -4-(N-methyl)pyridinium; R₂, R₃, R₅, R₆, R₈, R₉, R₁ and R₁₂ are each —H; X^{p} is Cl⁻; m is 5; and n is 5.

13. The method of claim 1, wherein R₁, R₄, R₇ and R₁₀ are each -4-sulfanatophenyl; R₂, R₃, R₅, R₆, R₈, R₉, R₁₁ and R₁₂ are each —H; X^{p} is Na⁺; m is +3; and n is 3.

14. A method for induction of apoptosis of cancer cells comprising administering to a patient in need thereof a composition comprising an effective amount of a gold(III) complex of formula: 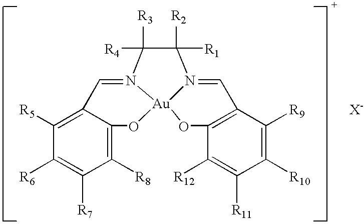
or a pharmaceutically acceptable salt thereof, wherein:
R₁-R₁₂ are each independently —H, -halo, —(C₁-C₆)alkyl or —O(C₁-C₆)alkyl which may be substituted with one or more —O(C₁-C₆)alkyl or -halo;
X is a counter-anion; and
a pharmaceutically acceptable carrier.

15. The method of claim 14, wherein R₁-R₄ are each —H; and X is Cl⁻.

16. The method of claim 15, wherein R₅-R₁₂ are each —H.

17. The method of claim 15, wherein R₅, R₇-R₉ and R₁₁-R₁₂ are each —H; and R₆ and R₁₀ are each —Cl.

18. The method of claim 15, wherein R₅, R₇, R₉ and R₁₀ are each —H; and R₆, R₈, R₁₀ and R₁₂ are each —Cl.

19. A method for induction of apoptosis of cancer cells comprising administering to a patient in need thereof a composition comprising an effective amount of a gold(III) complex of formula: 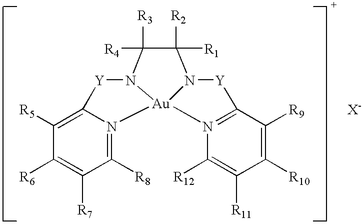
or a pharmaceutically acceptable salt thereof, wherein:
(a) R₁-R₁₂ are each independently —H, -halo, —(C₁-C₆)alkyl —O(C₆)alkyl which may be substituted with one or more —O(C₁-C₆)alkyl or -halo; or
(b) R₁ and R₄ are absent; and R₂ and R₃ together form a 6-membered aryl ring of formula 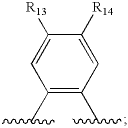
Y is 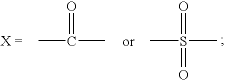
R₁₃ and R₁₄ are each —H or -halo;
X is a counter-anion; and
a pharmaceutically acceptable carrier.

20. The method of claim 19, wherein
Y is 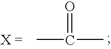
and
X is Cl⁻.

21. The method of claim 20, wherein R₁—R₁₂ are each —H.

22. The method of claim 20, wherein R₁-R₄ are each -methyl; and R₅-R₁₂ are each —H.

23. The method of claim 20, wherein R₁ and R₄-R₁₂ are each —H; and R₂ and R₃ are each -phenyl.

24. The method of claim 20, wherein R₁ and R₄ are absent; R₂ and R₃ together form 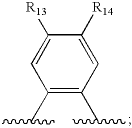
and
R₅-R₁₂ are each —H.

25. A method for inhibition of reverse transcriptase of Human Immunodeficiency virus-1 comprising administering to a patient in need thereof a composition comprising an effective amount of a gold(III) complex of formula: 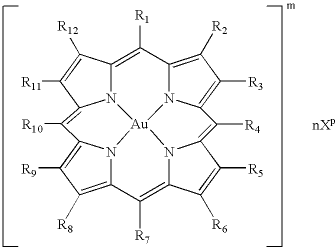
or a pharmaceutically acceptable salt thereof, wherein:
R₁, R₄, R₇ and R₁₀ are each independently —H, -halo, —(C₁-C₆)alkyl or —O(C₁-C₆)alkyl, -(6-membered)aryl or -(5 to 10-membered)heteroaryl, each of which may be substituted with one or more -halo, —(C₁-C₆)alkyl, —O(C₁-C₆)alkyl, —OSO₂ or —NO₂;
R₂, R₃, R₅,R₆, R₈, R₉, R₁₀, and R₁₂ are each independently —H, —(C₁-C₆)alkyl which may be substituted with one or more —C(O)OR₁₃, -halo or ═O groups;
R₁₃ is —(C₁-C₆)alkyl;
each X^{p} is independently a pharmaceutically acceptable counter-ion;
m is an integer ranging from −3 to 5;
p is an integer ranging from −3 to 3;
n is equal to the absolute value of m/p; and
a pharmaceutically acceptable carrier.

26. The method of claim 25, wherein R₂, R₃, R₅, R₆, R₈, R₉, R₁₁ and R₁₂ are each —H.; X^{p} is Cl⁻; m is 1; and n is 1.

27. The method of claim 26, wherein R₁, R₄, R₇ and R₁₀ are each -phenyl.

28. The method of claim 26, wherein R₁, R₄, R₇ and R₁₀ are each -4-methylphenyl.

29. The method of claim 26, wherein R₁, R₄, R₇ and R₁₀ are each -4-methoxyphenyl.

30. The method of claim 26, wherein R₁, R₄, R₇ and R₁₀ are each -4-bromophenyl.

31. The method of claim 26, wherein R₁, R₄, R₇ and R₁₀ are each -4-chlorophenyl.

32. The method of claim 26, wherein R₁, R₄, R₇ and R₁₀ are each -3,4,5-trimethoxyphenyl.

33. The method of claim 26, wherein R₁, R₄, R₇ and R₁₀ are each -3,4,5-trifluorophenyl.

34. The method of claim 25, wherein R₁, R₄, R₇ and R₁₀ are each —H; R₂, R₃, R₅, R₆, R₈, R₉, R₁₁ and R₁₂ are each -ethyl; X^{p} is Cl⁻; m is 1; and n is 1.

35. The method of claim 25, wherein R₁, R₄, R₇ and R₁₀ are each —H; and R₂ and R., are each -ethyl; R₃, R₅, R₉ and R₁₂ are each -methyl; R₆ and R₈ are each -methyl-3-propanoate; X^{p} is Cl⁻; m is 1; and n is 1.

36. The method of claim 25, wherein R₁, R₄, R₇ and R₁₀ are each -4-(N-methyl)pyridinium; R₂, R₃, R₅, R₆, R₈, R₉, R₁₁ and R₁₂ are each —H; X^{p} is Cl⁻; m is 5; and n is 5.

37. The method of claim 25, wherein R₁, R₄, R₇ and R₁₀ are each -4-sulfanatophenyl; R₂, R₃,R₅, R₆, R₈, R₉, R₁₁ and R₁₂ are each —H; X^{p} is Na⁺; m is =3; and n is S.

38. A method for inhibition of reverse transcriptase of Human Immunodeficiency virus-1 comprising administering to a patient in need thereof a composition comprising an effective amount of a gold(III) complex of formula: 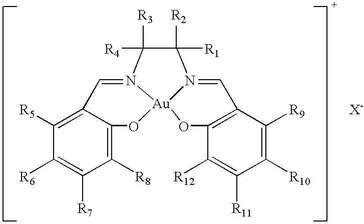
or a pharmaceutically acceptable salt thereof, wherein:
R₁-R₁₂ are each independently —H, -halo, —(C₁-C₆)alkyl or —O(C₁-C₆)alkyl which may be substituted with one or more —O(C₁-C₆)alkyl or -halo;
X is a counter-anion; and
a pharmaceutically acceptable carrier.

39. The method of claim 38, wherein R₁, R₁′, R₂ and R₂′ are each —H; and X is Cl⁻.

40. The method of claim 39, wherein R₃-R₁₀ are each —H.

41. The method of claim 38, wherein R₃, R₅-R₇ and R₉-R₁₀ are each —H; and R₄ and R₈ are each —Cl.

42. The method of claim 38, wherein R₃, R₅, R₇ and R₉ are each —H; and R₄, R₆, R₈ and R₁₀ are each —Cl.

43. A method for inhibition of reverse transcriptase of Human Immunodeficiency virus-1 comprising administering to a patient in need thereof a composition comprising an effective amount of a gold(III) complex of formula: 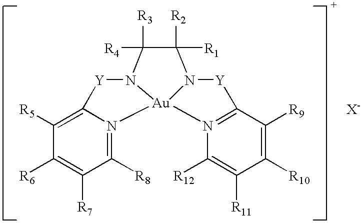
or a pharmaceutically acceptable salt thereof, wherein:
(a) R₁-R₁₂ are each independently —H, -halo, —(C₁-C₆)alkyl —O(C₆)alkyl which may be substituted with one or more —O(C₁-C₆)alkyl or -halo; or
(b) R₁ and R₄ are absent; and R₂ and R₃ together form a 6-membered aryl ring of formula 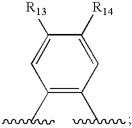
Y is 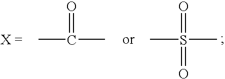
R₁₃ and R₁₄ are each —H or -halo;
X is a counter-anion; and
a pharmaceutically acceptable carrier.

44. The method of claim 43, wherein
Y is 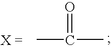
and
X is Cl⁻.

45. The method of claim 44, wherein R₁-R₁₂ are each —H.

46. The method of claim 44, wherein R₁-R₄ are each -methyl; and R₅-R₁₂ are each —H.

47. The method of claim 44, wherein R₁ and R₄-R₁₂ are each —H; and R₂ and R₃ are each -phenyl.

48. The method of claim 44, wherein R₁ and R₄ are absent; R₂ and R₃ together form 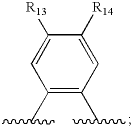
and
R₅-R₁₂ are each —H.

49. A pharmaceutical composition comprising an effective amount of a gold(III) complex of formula: 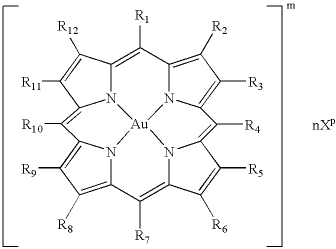
or a pharmaceutically acceptable salt thereof, wherein:
R₁, R₄, R₇ and R₁₀ are each independently —H, -halo, —(C₁-C₆)alkyl or —O(C₁-C₆)alkyl, -(6-membered)aryl or -(5 to 10-membered)heteroaryl, each of which may be substituted with one or more -halo, —(C₁-C₆)alkyl, —O(C₁-C₆)alkyl, —OSO₂ or —NO₂;
R₂, R₃, R₅, R₆, R₈, R₉, R₁₁ and R₁₂ are each independently —H, —(C₁-C₆)alkyl which may be substituted with one or more —C(O)OR₁₃, -halo or ═O groups;
R₁₃ is —(C₁-C₆)alkyl;
each X^{p} is independently a pharmaceutically acceptable counter-ion;
m is an integer ranging from −3 to 5;
p is an integer ranging from −3 to 3;
n is equal to the absolute value of m/p; and
a pharmaceutically acceptable carrier.

50. The composition of claim 49 further comprising 3′-azido-2′,3′-dideoxythymidine.

51. A pharmaceutical composition comprising an effective amount of a gold(III) complex of formula: 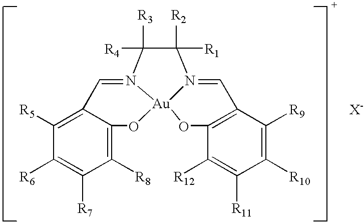
or a pharmaceutically acceptable salt thereof, wherein:
R₁-R₁₂ are each independently —H, -halo, —(C₁-C₆)alkyl or —O(C₁-C₆)alkyl which may be substituted with one or more —O(C₁-C₆)alkyl or -halo;
X is a counter-anion; and
a pharmaceutically acceptable carrier.

52. The composition of claim 51 further comprising 3′-azido-2′,3′-dideoxythymidine.

53. A pharmaceutical composition comprising an effective amount of a gold(III) complex of formula: 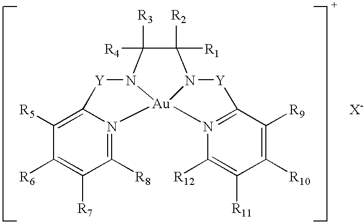
or a pharmaceutically acceptable salt thereof, wherein:
(a) R₁-R₁₂ are each independently —H, -halo, —(C₁-C₆)alkyl —O(C₆)alkyl which may be substituted with one or more —O(C₁-C₆)alkyl or -halo; or
(b) R₁ and R₄ are absent; and R₂ and R₃ together form a 6-membered aryl ring of formula 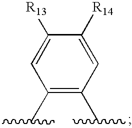
Y is 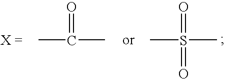
R₁₃ and R₁₄ are each —H or -halo;
X is a counter-anion; and
a pharmaceutically acceptable carrier.

54. The composition of claim 53 further comprising 3′-azido-2′,3′-dideoxythymidine.

55. A method for inhibition of reverse transcriptase of Human Immunodeficiency virus-1 comprising administering to a patient in need thereof a composition comprising an effective amount of a gold(III) complex of claim 50.

56. A method for inhibition of reverse transcriptase of Human Immunodeficiency virus-1 comprising administering to a patient in need thereof a composition comprising an effective amount of a gold(III) complex of claim 52.

57. A method for inhibition of reverse transcriptase of Human immunodeficiency virus-1 comprising administering to a patient in need thereof a composition comprising an effective amount of a gold(III) complex of claim 54.

58. A complex formed between a ligand and a gold(III) complex of formula: 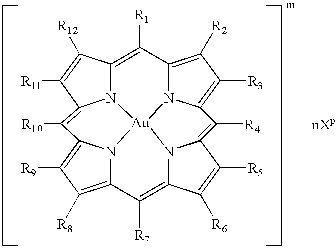
or a pharmaceutically acceptable salt thereof, wherein:
R₁, R₄, R₇ and R₁₀ are each independently —H, -halo, —(C₁-C₆)alkyl or —O(C₁-C₆)alkyl, -(6-membered)aryl or -(5 to 10-membered)heteroaryl, each of which may be substituted with one or more -halo, —(C1-C₆)alkyl, —O(C₁-C₆)alkyl, —OSO₂ or —NO₂;
R₂, R₃, R₅, R₆, R₈, R₉, R₁₁ and R₁₂ are each independently —H, —(C₁-C₆)alkyl which may be substituted with one or more —C(O)OR₁₃, -halo or ═O groups;
R₁₃ is —(C₁-C₆)alkyl;
each X^{p} is independently a pharmaceutically acceptable counter-ion;
m is an integer ranging from −3 to 5;
p is an integer ranging from −3 to 3; and
n is equal to the absolute value of m/p.

59. The complex of claim 58, wherein the ligand is selected from the group consisting of porphyrins, metalloporphyrins, amino acids, peptides, polypeptides, proteins, nucleotides, polynucleotides, deoxyribonucleic acid, and ribonucleic acid.

60. A complex formed between a ligand and a gold(III) complex of formula: 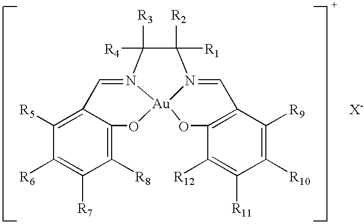
or a pharmaceutically acceptable salt thereof, wherein:
R₁-R₁₂ are each independently —H, -halo, —(C₁-C₆)alkyl or —O(C₁-C₆)alkyl which may be substituted with one or more —O(C₁-C₆)alkyl or -halo; and
X is a counter-anion.

61. The complex of claim 60, wherein the ligand is selected from the group consisting of porphyrins, metalloporphyrins, amino acids, peptides, polypeptides, proteins, nucleotides, polynucleotides, deoxyribonucleic acid, and ribonucleic acid.

62. A complex formed between a ligand and a gold(III) complex of formula: 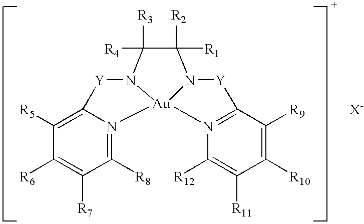
or a pharmaceutically acceptable salt thereof, wherein:
(a) R₁-R₁₂ are each independently —H, -halo, —(C₁-C₆)alkyl —O(C₆)alkyl which may be substituted with one or more —O(C₁-C₆)alkyl or -halo; or
(b) R₁ and R₄ are absent; and R₂ and R₃ together form a 6-membered aryl ring of formula 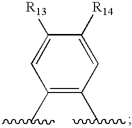
Y is 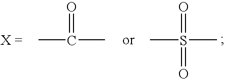
R₁₃ and R₁₄ are each —H or -halo; and
X is a counter-anion.

63. The complex of claim 62, wherein the ligand is selected from the group consisting of porphyrins, metalloporphyrins, amino acids, peptides, polypeptides, proteins, nucleotides, polynucleotides, deoxyribonucleic acid, and ribonucleic acid.
